(19) United States
(12) Patent Application Publication
(10) Pub. No.: US 2013/0040952 A1
(43) Pub. Date: Feb. 14, 2013

(54) INFLUENZA VIRUS INHIBITORS THAT DISRUPT NUCLEOPROTEIN TRIMERIZATION

(71) Applicants: Chi-Huey Wong, Rancho Santa Fe, CA (US); Ming-Daw Tsai, Taipei (TW); Ying-Ta Wu, Taipei (TW); Yih-Shyun E. Cheng, Taipei (TW); Yu-Hou Chen, New Taipei City (TW); Yu-Fang Shen, Taipei City (TW)

(72) Inventors: Chi-Huey Wong, Rancho Santa Fe, CA (US); Ming-Daw Tsai, Taipei (TW); Ying-Ta Wu, Taipei (TW); Yih-Shyun E. Cheng, Taipei (TW); Yu-Hou Chen, New Taipei City (TW); Yu-Fang Shen, Taipei City (TW)

(73) Assignee: Academia Sinica, Taipei (TW)

(21) Appl. No.: 13/567,355

(22) Filed: Aug. 6, 2012

Related U.S. Application Data
(60) Provisional application No. 61/515,301, filed on Aug. 4, 2011.

(51) Int. Cl. A61K 31/5377; C12N 5/07; A61P 31/16; G01N 33/53; A61K 31/167; A61K 31/517

(52) U.S. Cl. 514/236.8; 514/616; 514/258.1; 435/6.19; 435/325

(57) ABSTRACT

Methods for identifying agents capable of disrupting a salt bridge in an influenza A virus nucleoprotein corresponding to the E339 . . . R416 salt bridge in SEQ ID NO:1, and thus the trimerization of the NP protein; and uses of such agents, e.g., small molecules and peptides, for inhibiting influenza virus replication and treating infection caused by influenza virus.

### RELATED APPLICATIONS

This application claims priority under 35 U.S.C. §119 to U.S. provisional application 61/515,301, filed Aug. 4, 2011, the entire content of which is herein incorporated by reference in its entirety.

### BACKGROUND OF THE INVENTION

Outbreaks of influenza virus infection cause widespread morbidity and mortality worldwide. In the United States, an estimated 5 to 20% of the population is infected by influenza A virus annually, causing approximately 200,000 hospitalizations and 36,000 deaths. As influenza viruses have developed resistance towards current drugs, new inhibitors that prevent viral replication through different inhibitory mechanisms are of great importance.

Currently available drugs for treating influenza virus infection include M2 channel blockers such as amantidine and rimantidine (Margo K L, et al. (1998) Am Fam Physician 57:1073-1077), and neuraminidase inhibitors such as Oseltamivir and Zanamivir (Oxford J S, et al., (2003) Expert Rev Anti Infe 1:337-342; Oxford J, et al., (2004) J Antimicrob Chemother 53:133-136). However, high percentages of circulating influenza strains have developed resistance to amantidine and rimantidine (Monto A S, et al. (1992) Clin Infect Dis 15:362-367; Deyde V M, et al. (2007) J Infect Dis 196:249-257). Viruses that are resistant to the neuraminidase inhibitor Oseltamivir have also been reported since 2005 (de Jong M D, et al. (2005) N Engl J Med 353:2667-2672; Lackenby A, et al. (2008) Eurosurveillance 13:1-2), and currently greater than 75% of influenza H1N1 viruses in Norway and many other countries are resistant to Oseltamivir (Hauge S H, et al. (2009) Emerg Infect Dis 15:155-162).

Given the development of drug resistance in treating influenza viruses, it is of great interest to develop new anti-influenza drugs that are effective in inhibiting a broad spectrum of influenza viruses.

### SUMMARY OF THE INVENTION

The present disclosure is based on the unexpected discovery that (a) the E339 . . . R416 salt bridge in the influenza A virus nucleoprotein (SEQ ID NO:1, a representative viral NP protein) is essential in viral replication and thus a useful target for treating influenza virus infection, and (b) a number of compounds and peptides capable of disrupting this salt bridge effectively blocked influenza A virus replication. These findings indicate that the viral nucleoprotein (NP), particularly the just-noted salt bridge therein, can serve as a target for treating influenza virus infection and identifying anti-flu agents.

Accordingly, the present disclosure provides methods for identifying influenza virus inhibitors capable of blocking formation of NP trimers by disrupting a salt bridge in an influenza virus nucleoprotein, wherein the salt bridge corresponds to the E339 . . . R416 salt bridge in SEQ ID NO:1, compounds (small molecules) and peptides capable of inhibiting viral replication via, e.g., disrupting the salt bridge in the NP protein, and compositions containing such for inhibiting viral replication and/or treating influenza virus infection.

One aspect of the present disclosure features a screening method of identifying influenza A virus inhibitors, the method comprising (1) contacting a candidate agent with an influenza A virus nucleoprotein, which is in trimer form, (2) determining the disruption level of the trimer form of the nucleoprotein by the candidate agent, and (3) assessing whether the candidate agent is an influenza A virus inhibitor. If the candidate agent disrupts the trimer form of the nucleoprotein, it is identified as an inhibitor of the influenza A virus.

In some embodiments, the determining step is performed by detecting presence of monomers or oligomers (which are not trimers, i.e., containing less than or more than 3 monomers) of the nucleoprotein after the contacting step. Presence of the monomers/oligomers can be detected by a process comprising: (a) performing an analytical ultracentrifugation (AUC) assay on the nucleoprotein after the trimer nucleoprotein is contacted with the test agent, (b) measuring mass distribution of the nucleoprotein; and (c) comparing the mass distribution with that of the nucleoprotein in trimer form. If the mass distribution of the nucleoprotein treated with the candidate agent differs from that of the nucleoprotein in trimer form, it indicates presence of the monomers/oligomers of the nucleoprotein, which, in turn, indicates disruption of the trimer form of the nucleoprotein.

In another aspect, the present disclosure provides methods of inhibiting replication of an influenza virus (e.g., influenza A virus) and/or treating influenza virus infection. The method comprises contacting cells infected or suspected of being infected with the virus (e.g., by administering to a subject in need thereof) with an effective amount of an agent that inhibits viral replication via, e.g., disrupting a salt bridge in the nucleoprotein of the influenza virus, wherein the salt bridge corresponds to the E339 . . . R416 salt bridge in SEQ ID NO:1. Such an agent includes any of the compounds/peptides disclosed herein, and agents (e.g., small molecules, peptides, oligonucleotides, oligosaccharides) identified in the screening methods also disclosed herein. The subject in need of the treatment can be a human patient has or is suspected of having infection with a wild-type influenza A virus (e.g., H1N1, H5N1, or H3N2) or with a mutant influenza virus, such as one that has a mutated NP protein, e.g., an NP protein having the Y289H, Y52H, or Y52H/Y289H mutations.

In some embodiments, the agent used in the above-described method is a compound of formula (I):

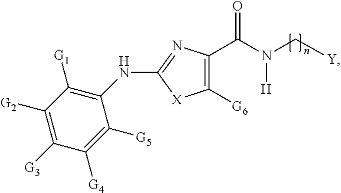

in which each of G₁, G₂, G₃, G₄, G₅ and G₆ is, independently, H, F, Cl, Br, I, OH, O-alkyl (C₁-C₃), NH₂, NH-alkyl (C₁-C₂), NR₂ (NR′R″, in which each of R′ and R″ is independently CH₃ or C₂H₅), NHCOR(R═CH₃ or C₂H₅), N₃, NO₂, alkyl (C1-C3), CF₃, phenyl, C≡N, CHO, RCO(R═CH₃ or C₂H₅), CO₂H, CO₂R(R═CH₃ or C₂H₅), CONHR(R═CH₃ or C₂H₅), or SO₃H; X is S, O, NH, or NR(R═CH₃ or C₂H₅); n is any integer between 1 and 6 inclusive (1, 2, 3, 4, 5, or 6); and Y is a terminal group selected from phenyl (which can be substituted), morpholine, and piperazine (which can be substituted). In one example, the compound of formula (I) is Compound 1:

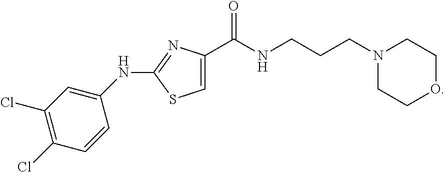 [SMILES: O=C(NCCCN1CCOCC1)c1csc(Nc2ccc(Cl)c(Cl)c2)n1]

In other embodiments, the agent used in the method described herein is a compound of formula (II):

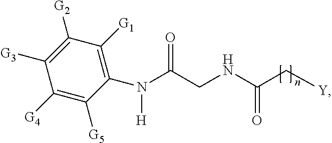

in which each of G₁, G₂, G₃, G₄ and G₅, independently, is H, F, Cl, Br, I, OH, O-alkyl (C₁-C₃), NH₂, NH-alkyl (C₁-C₂), NR₂ (NR′R″, in which each of R′ and R″ is independently CH₃ or C₂H₅), NHCOR(R═CH₃ or C₂H₅), N₃, NO₂, alkyl (C₁-C₃), CF₃, phenyl, C≡N, CHO, RCO(R═CH₃ or C₂H₅), CO₂H, CO₂R(R═CH₃ or C₂H₅), CONHR(R═CH₃ or C₂H₅), or SO₃H; n is any integer between 1 and 10 inclusive; and Y is alkyl or aryl. In one example, the compound of formula (II) is:

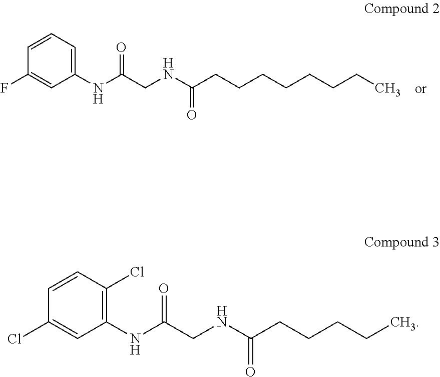 [SMILES: CCCCCCCCC(=O)NCC(=O)Nc1cccc(F)c1 ; CCCCCC(=O)NCC(=O)Nc1cc(Cl)ccc1Cl]

In still some embodiments, the agent used in the method described herein is a compound of formula (III):

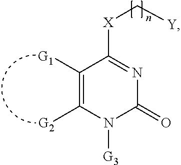

in which each of G₁ and G₂ is, independently, H, F, Cl, Br, I, OH, O-alkyl (C₁-C₃), NH₂, NH-alkyl (C₁-C₂), NR₂ (NR′R″, in which each of R′ and R″ is independently CH₃ or C₂H₅), NHCOR(R═CH₃ or C₂H₅), N₃, NO₂, alkyl (C₁-C₃), CF₃, phenyl, C≡N, CHO, CH₃CO, CO₂H, CO₂R(R═CH₃ or C₂H₅), CONHR(R═CH₃ or C₂H₅), or SO₃H, and G₁ and G₂ can be connected to form a 5-7 membered ring; G₃ is H, alkyl (C₁-C₆), [phenyl (e.g., substituted)]methyl, ω-hydroxyalkyl (C₁-C₄), phenyl (e.g., substituted), RCO(R═CH₃ or C₂H₅), CO₂R(R═CH₃ or C₂H₅), or CONR₂ (NR′R″, in which each of R′ and R″ is independently CH₃ or C₂H₅); X is CH₂, S, O, NH, or NR(R═CH₃ or C₂H₅); n is any integer between 1 and 6 inclusive; and Y is a terminal group selected from phenyl (which can be substituted), morpholine, and piperazine (which can be substituted). In one example, the compound of formula (III) is:

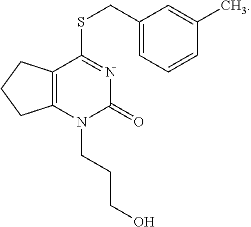 [SMILES: Cc1cccc(CSc2nc(=O)n(CCCO)c3c2CCC3)c1]

In yet other embodiments, the agent used in the method described herein can be a compound of formula (IV):

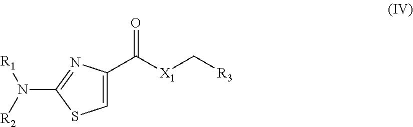

wherein:

X₁ is selected from the group consisting of CR_{d}Rₑ, NR_{f}, C═O, O and S, wherein each occurrence of R_{d}, Rₑ and R_{f} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety;

each instance of R₁ and R₂ is, independently, hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, or —NR_{b}R_{c}, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety;

R₃ is hydrogen, alkyl, alkenyl, alkynyl, cycloalkyl, cycloalkenyl, heterocycloalkyl, heterocycloalkenyl, aryl, heteroaryl, halo, nitro, —CN, —COORₐ, —C(═O)Rₐ, —C(═O)NR_{b}R_{c}, —ORₐ, —OC(═O)Rₐ, —OC(═O)ORₐ, —OC(═O)N(Rₐ)₂, —N₃, —NR_{b}R_{c}, —NHC(═O)Rₐ, —NRₐ C(═O)NR_{b}R_{c}, —NRₐ C(═O)ORₐ, —SCN, —SRₐ, —S(═O)Rₐ, —S(═O)₂Rₐ, or an amino protecting group, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety.

Alternatively, the compound used in the method described herein is a compound of formula (V):

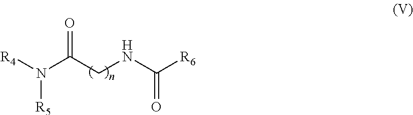

wherein:

n is an integer between 1 and 6, inclusive;

each instance of R₄ and R₅ is, independently, hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, —NR_{b}R_{c}, wherein each occurrence of Rₐ, R_{b}, and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety;

R₃ is hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, —ORₐ, —OC(═O)Rₐ, —OC(═O)ORₐ, —OC(═O)N(Rₐ)₂, —NR_{b}R_{c}, —NHC(═O)Rₐ, —NRₐ C(═O)NR_{b}R_{c}, —NRₐ C(═O)ORₐ, —SRₐ, or —S(═O)Rₐ, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety.

The agent used in the method described herein can also be a compound of formula (VI):

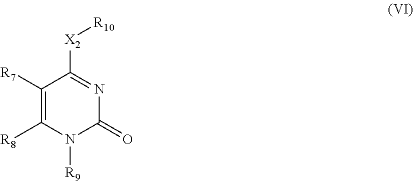

wherein:

X₂ is selected from the group consisting of CR_{d}Rₑ, NR_{f}, C═O, O and S, wherein each occurrence of R_{d}, Rₑ and R_{f} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or heteroarylthio moiety;

each instance of R₇, R₈ and R₁₀ is, independently, hydrogen, alkyl, alkenyl, alkynyl, cycloalkyl, cycloalkenyl, heterocycloalkyl, heterocycloalkenyl, aryl, heteroaryl, halo, nitro, —CN, —COORₐ, —C(═O)Rₐ, —C(═O)NR_{b}R_{c}, —ORₐ, —OC(═O)Rₐ, —OC(═O)ORₐ, —OC(═O)N(Rₐ)₂, —N₃, —NR_{b}R_{c}, —NHC(═O)Rₐ, —NRₐ C(═O)NR_{b}R_{c}, —NRₐ C(═O)ORₐ, —SCN, —SRₐ, —S(═O)Rₐ, —S(═O)₂Rₐ, or an amino protecting group, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety; and

R₉ is hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, —NR_{b}R_{c}, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety.

Unless specifically pointed out, alkyl, alkenyl, alkynyl, aryl, heteroaryl, cyclyl, and heterocyclyl mentioned herein include both substituted and unsubstituted moieties. The term “substituted” refers to one or more substituents (which may be the same or different), each replacing a hydrogen atom. Examples of substituents include, but are not limited to, halogen, cyano, nitro, hydroxyl, amino, mercapto, alkyl, alkenyl, alkynyl, aryl, heteroaryl, cyclyl, heterocyclyl, alkyloxy, aryloxy, alksulfanyl, arylsulfanyl, alkylamino, arylamino, dialkylamino, diarylamino, alkylcarbonyl, arylcarbonyl, heteroarylcarbonyl, alkylcarboxyl, arylcarboxyl, heteroarylcarboxyl, alkyloxycarbonyl, aryloxycarbonyl, heteroaryloxycarbonyl, alkylcarbamido, arylcarbamido, heterocarbamido, alkylcarbamyl, arylcarbamyl, heterocarbamyl, wherein each of alkyl (including alk), alkenyl, aryl, heteroaryl, cyclyl, and heterocyclyl is optionally substituted with halogen, cyano, nitro, hydroxyl, amino, mercapto, alkyl, aryl, heteroaryl, alkyloxy, aryloxy, alkylcarbonyl, arylcarbonyl, alkylcarboxyl, arylcarboxyl, alkyloxycarbonyl, or aryloxycarbonyl.

All of the compounds described herein, including the compounds of formulae (I)-(VI) such as compounds 1, 2, 3 and 4, include the compounds themselves, as well as their salts, their solvates, and their prodrugs, if applicable. A salt, for example, can be formed between an anion and a positively charged group (e.g., amino) on a compound described herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate. Likewise, a salt can also be formed between a cation and a negatively charged group (e.g., carboxylate) on a compound described herein. Suitable cations include sodium ion, potassium ion, magnesium ion, calcium ion, and an ammonium cation such as tetramethylammonium ion. The compounds described herein also include those salts containing quaternary nitrogen atoms. Examples of prodrugs include esters and other pharmaceutically acceptable derivatives, which, upon administration to a subject, are capable of providing active compounds to inhibit influenza virus infection.

In other embodiments, the agent used in the method described herein is an isolated peptide comprising an amino acid sequence at least 80% (e.g., 85%, 90%, 95%, or 98%) identical to an influenza A virus NP fragment that encompasses an Arg residue corresponding to the R416 residue in SEQ ID NO:1. In one example, the NP fragment, or a portion thereof, corresponds to the region of residues 402-428 in SEQ ID NO:1. In another example, the fragment corresponds to the region spanning from T411 to N417 of SEQ ID NO:1. When necessary, the peptide can include two cysteine residues flanking the N-terminus and C-terminus of the NP fragment. In that case, the peptide can be cyclized through a disulfide bond between the two cysteine residues. In other embodiments, the peptide, either linear or cyclic, comprises the amino acid sequence of CTFSVQRNC (SEQ ID NO:2), CPTFSVQRNLC (SEQ ID NO:3), or CQPTFSVQRNLC (SEQ ID NO:4).

Any of the compounds and peptides (in isolated form) described herein, as well as a composition (e.g., a pharmaceutical composition comprising the compound or peptide and a pharmaceutically acceptable carrier), is also within the scope of the present disclosure. An isolated peptide refers to a peptide or polypeptide substantially free from naturally associated molecules, i.e., the naturally associated molecules constituting at most 20% by dry weight of a preparation containing the polypeptide. Purity can be measured by any appropriate method, e.g., column chromatography, polyacrylamide gel electrophoresis, and HPLC.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1 shows IP assays of protein-protein interactions in cells. (A) Interaction of NP-Flag with NP-HA or mutant-HA. (B) AUC analyses to examine the ability of the NP mutants to mix with WT NP. (C) Pull-down assays for the interaction of NP proteins and PB1 (D) The transcription-replication activity is reduced by NP mutant proteins.

FIG. 2 shows the effects of the tail-loop peptide 402-428. (A) Interaction of NP WT and EGFP-fused tail-loop peptide. (B) Luciferase-based reporter assays of the effect of EGFP-tail-loop on the H1N1 polymerase activity. (C) The inhibition effect of EGFP-fused tail-loop peptide (H1N1) on viral replication. (D) HEK293T cells which were expressed with GFP or GFP-TL were infected with the WSN virus (E) AUC analyses of the tail-loop peptide

FIG. 3 shows the effects of short cyclic peptides. (A) Analytical ultracentrifugation (AUC) analyses for NP WT mass distribution free and in the presence of a linear short peptide (peptide 1) and its circularized forms (peptide 2 and 3). (B) The antiviral dose response curves of the peptides. (C) The inhibition effects in the in vitro transcription assay.

FIG. 4 shows the effects of compounds for NP WT trimer by AUC.

FIG. 5 shows the inhibition effects of small molecule inhibitors. (A) Structures and inhibitory effects of the compounds. (B) In vitro transcription assay of compounds. (C) The antiviral dose response curves of compound 1. (D) The modeled structure of the NP-compound 1 complex.

### DETAILED DESCRIPTION OF THE INVENTION

Influenza A virus is an RNA virus, which requires an RNA-dependent RNA polymerase (RDRP) for its replication. RDRP is a protein complex composed of polymerase basic protein 1 (PB1), basic protein 2 (PB2), and acidic protein (PA) (Neumann G, et al. (2009) Nature 459(7249):931-939). To be functional, RDRP must be associated with the viral nucleoprotein (NP) to form a ribonucleoprotein (RNP) complex. (Coloma R, et al. (2009) PLoS Pathog 5(6):e1000491).

Provided below is the amino acid sequence of an exemplary influenza viral NP protein, which has 498 amino acid residues (SEQ ID NO:1).

```
                10 20 30 40 50 60
MASQGTKRSY EQMETDGERQ NATEIRASVG KMIGGIGRFY IQMCTELKLS DYEGRLIQNS
                70 80 90 100 110 120
LTIERMVLSA FDERRNKYLE EHPSAGKDPK KTGGPIYRRV NGKWMRELIL YDKEEIRRIW
              130 140 150 160 170 180
RQANNGDDAT AGLTHMMIWH SNLNDATYQR TRALVRTGMD PRMCSLMQGS TLPRRSGAAG
              190 200 210 220 230 240
AAVKGVGTMV MELVRMIKRG INDRNFWRGE NGRKTRIAYE RMCNILKGKF QTAAQKAMMD
              250 260 270 280 290 300
QVRESRNPGN AEFEDLTFLA RSALILRGSV AHKSCLPACV YGPAVASGYD FEREGYSLVG
              310 320 330 340 350 360
IDPFRLLQNS QVYSLIRPNE NPAHKSQLVW MACHSAAFED LRVLSFIKGT KVLPRGKLST
              370 380 390 400 410 420
RGVQIASNEN METMESSTLE LRSRYWAIRT RSGGNTNQQR ASAGQISIQP TFSVQRNLPF
              430 440 450 460 470 480
DRTTVMAAFS GNTEGRTSDM RTEIIRMMES ARPEDVSFQG RGVFELSDEK AASPIVPSFD
              490
MSNEGSYFFG DNAEEYDN
```

Crystal structures of NP indicate that, in nature, this protein exists in trimer form and its tail-loop region (corresponding to residues 402 to 428 in SEQ ID NO:1) plays an important role in NP trimerization. This trimer form of NP is essential to its association with RDRP to form the RNP complex noted above. Thus, disrupting the trimer form of NP would be effective in blocking the formation of the RNP complex and, consequently, inhibiting influenza viral replication. As NP trimerization and formation of the RNP complex are processes essential to the replication of all influenza virus species, targeting NP trimerization via, e.g., disrupting the salt bridge noted herein, would be effective in blocking replication of a broad spectrum of influenza viruses and treating infections caused thereby.

The present disclosure is based on the unexpected discovery that the E339 . . . R416 salt bridge of the viral NP protein is essential to trimerization of the NP protein, which is necessary in formation of the viral replication complex, and that a number of compounds and peptides capable of disrupting this salt bridge effectively suppressed NP protein trimerization and in turn, blocked viral replication. Accordingly, the salt bridge of a viral NP protein corresponding to the E339 . . . R416 salt bridge of SEQ ID NO:1 can serve as a useful target in treatment of influenza infection and identification of anti-flu agents.

Accordingly, disclosed herein are methods for identifying anti-influenza virus agents that disrupts the salt bridge in a viral NP protein corresponding to the E339 . . . R416 salt bridge in SEQ ID NO:1, pharmaceutical compositions comprising such anti-influenza virus agents, and uses thereof for inhibiting viral replication and/or treating viral infection.

### I. Methods for Identifying Anti-Influenza Virus Agents

The present disclosure provides a screening method for identifying influenza A virus inhibitors capable of disrupting NP trimer formation, which may be achieved by disrupting the salt bridge of that NP corresponding to the E339 . . . R416 salt bridge in SEQ ID NO:1. To perform this method, a candidate agent can be incubated with an NP protein, which is in trimer form, for a suitable period (e.g., 4 to 6 hr) under suitable conditions (e.g., 25° C.). The NP proteins used in this method can be prepared by recombinant technology and incubated under conditions suitable for trimer formation, which are within the knowledge of a skilled person in the art. After being incubated with the candidate agent, the NP protein can be examined to determine whether its trimer form has been disrupted via a routine method. In some examples, disruption of the trimer form can be indicated by presence of NP monomers and/or oligomers containing either less than three or more than three monomers.

In one example, the mass distribution of the NP proteins is determined by, e.g., an analytical ultracentrifugation assay as described in the Examples below. The results thus obtained can be compared with the mass distribution of NP trimmers determined by the same method. If the mass distribution of the NP proteins after the incubation with the candidate agent differs from that of NP trimers, it indicates that the candidate agent is capable of disrupting NP trimmers and therefore is an inhibitor of influenza A virus.

An agent capable of disrupting NP trimer formation can be an agent that reduces the formation of NP trimers by at least 20% (e.g., 30%, 40%, 50%, 60%, 70%, 80%, 90%, 95%, or 98%) after its incubation with the NP trimers as relative to prior incubation. When presence of NP monomer/oligomer containing either less than three or more than three monomers is used as an indicator for trimer disruption, an agent capable of disrupting NP trimer formation can either increase the level of such NP monomer/oligomer by at least 20% (e.g., 30%, 40%, 50%, 60%, 70%, 80%, 90%, 95%, or 98%) or decrease the level of NP trimers by at least 20% (e.g., 30%, 40%, 50%, 60%, 70%, 80%, 90%, 95%, or 98%) after its incubation with the NP trimers.

The inhibitory activity of any of the inhibitors identified from the screening methods described herein can be further confirmed by both in vitro and in vivo methods known in the art, such as those described in the Examples below.

### II. Anti-Influenza Virus Agents

The anti-influenza virus agents disclosed herein, including compounds and peptides, are capable of inhibiting influenza viral replication via disruption of NP trimer formation, which may be achieved by disrupting the salt bridge in a viral NP corresponding to E339 . . . R416 salt bridge in SEQ ID NO:1.

### (i) Compounds

Compounds disclosed herein that are capable of disrupting formation of NP trimers include those having the structures of Formula (I), Formula (II), and Formula (III) listed in Table 1 below, as well as Formula (IV), Formula (V), and Formula (VI) described herein. Examples of such compounds include, but are not limited to, Compounds 1, 2, 3, and 4, the structures of which are also shown in Table 1.

TABLE 1
Compounds of influenza virus inhibitors

| Compound | | Example | |
|---|---|---|---|
| 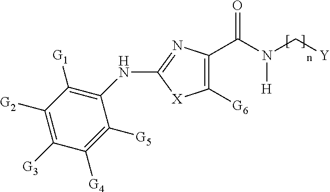 | For- mu- la I | 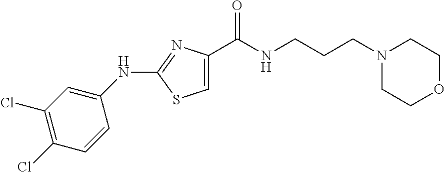 [SMILES: O=C(NCCCN1CCOCC1)c1csc(Nc2ccc(Cl)c(Cl)c2)n1] | Com- pound 1 |
| 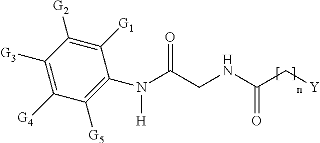 | For- mu- la II | 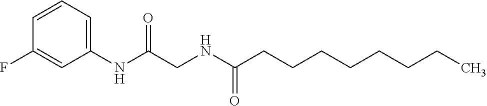 [SMILES: CCCCCCCCC(=O)NCC(=O)Nc1cccc(F)c1] | Com- pound 2 |
| | | 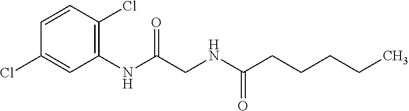 [SMILES: CCCCCC(=O)NCC(=O)Nc1cc(Cl)ccc1Cl] | Com- pound 3 |
| 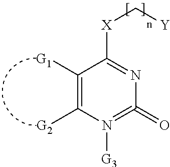 | For- mu- la III | 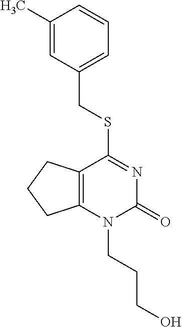 [SMILES: Cc1cccc(CSc2nc(=O)n(CCCO)c3c2CCC3)c1] | Com- pound 4 |

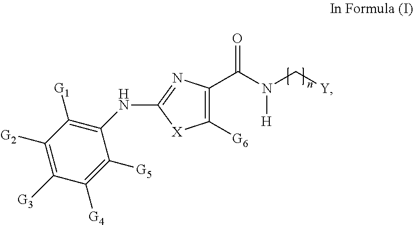

each of G₁, G₂, G₃, G₄, G₅ and G₆ can be a substituent independently selected from H, F, Cl, Br, I, OH, O-alkyl (C₁-C₃), NH₂, NH-alkyl (e.g., C₁-C₂ alkyl), NR₂ (R═CH₃ or C₂H₅), NHCOR(R═CH₃ or C₂H₅), N₃, NO₂, alkyl (e.g., C₁-C₃ alkyl), CF₃, phenyl, C≡N, CHO, RCO(R═CH₃ or C₂H₅), CO₂H, CO₂R(R═CH₃ or C₂H₅), CONHR(R═CH₃ or C₂H₅), and SO₃H; X is S, O, NH or NR(R═CH₃ or C₂H₅); n is an integer between 1 and 6, inclusive (i.e., 1, 2, 3, 4, 5, or 6); and Y is a terminal substituent selected from phenyl (which can be substituted), morpholine, and piperazine (which can be substituted). In some examples, at least one of G₁-G₆ (e.g., 2 of them) is H, F, Cl, Br, I (e.g., Cl). In other examples, X is O, S, or NH.

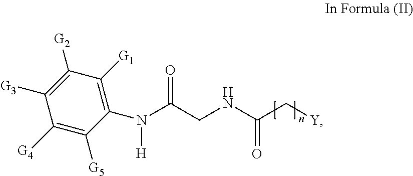

each of G₁, G₂, G₃, G₄ and G₅ can be a substituent independently selected from H, F, Cl, Br, I, OH, O-alkyl (e.g., C₁-C₃ alkyl), NH₂, NH-alkyl (e.g., C₁-C₂ alkyl), NR₂ (R═CH₃ or C₂H₅), NHCOR(R═CH₃ or C₂H₅), N₃, NO₂, alkyl (e.g., C₁-C₃ alkyl), CF₃, phenyl, C≡N, CHO, RCO (R═CH₃ or C₂H₅), CO₂H, CO₂R(R═CH₃ or C₂H₅), CONHR(R═CH₃ or C₂H₅), and SO₃H; n is an integer between 1 and 10, inclusive; and Y is a terminal group of alkyl or aryl. In one example, at least one of G₁ to G₆ is Cl or F. In an other example, Y is an alkyl group such as —CH₃.

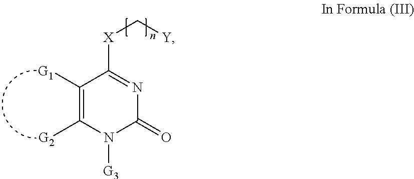

each of G₁ and G₂ can be a substituent independently selected from H, F, Cl, Br, I, OH, O-alkyl (e.g., C₁-C₃), NH₂, NH-alkyl (e.g., C₁-C₂ alkyl), NR₂ (R═CH₃ or C₂H₅), NHCOR(R═CH₃ or C₂H₅), N₃, NO₂, alkyl (e.g., C₁-C₃ alkyl), CF₃, phenyl, C≡N, CHO, CH₃CO, CO₂H, CO₂R(R═CH₃ or C₂H₅), CONHR(R═CH₃ or C₂H₅), and SO₃H; G₃ can be a substituent of H, alkyl (e.g., C₁-C₆ alkyl), [phenyl (e.g., substituted)]methyl, ω-hydroxyalkyl (e.g., C₁-C₄), ω-hydroxyalkyl (e.g., C₁-C₄), phenyl (e.g., substituted), RCO(R═CH₃ or C₂H₅), CO₂R(R═CH₃ or C₂H₅), and CONR₂ (R═CH₃ or C₂H₅); X can be CH₂, S, O, NH and NR(R═CH₃ or C₂H₅); n can be an integer between 1 and 6, inclusive; and Y is a terminal substituent which can be selected from phenyl (e.g., substituted), morpholine, and piperazine (e.g., substituted). In some examples, G₁ and G₂ (e.g., alkyl groups) can be connected to form a 5-7 membered ring. In other examples, X is O or S. In yet other examples, G₃ is a ω-hydroxyalkyl group which preferably is a C₁-C₄ hydroxyalkyl group.

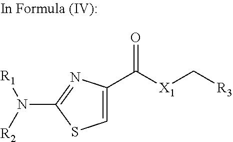

X₁ can be CR_{d}Rₑ, NR_{f}, C═O, O and S, wherein each occurrence of R_{d}, Rₑ and R_{f} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety;

each instance of R₁ and R₂ is, independently, hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, or —NR_{b}R_{c}, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety; and

R₃ is hydrogen, alkyl, alkenyl, alkynyl, cycloalkyl, cycloalkenyl, heterocycloalkyl, heterocycloalkenyl, aryl, heteroaryl, halo, nitro, —CN, —COORₐ, —C(═O)Rₐ, —C(═O)NR_{b}R_{c}, —ORₐ, —OC(═O)Rₐ, —OC(═O)ORₐ, —OC(═O)N(Rₐ)₂, —N₃, —NR_{b}R_{c}, —NHC(═O)Rₐ, —NRₐ C(═O)NR_{b}R_{c}, —NRₐ C(═O)ORₐ, —SCN, —SRₐ, —S(═O)Rₐ, —S(═O)₂Rₐ, or an amino protecting group, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety.

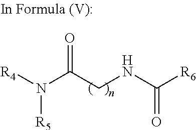

n is an integer between 1 and 6, inclusive;

each instance of R₄ and R₅ is, independently, hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, —NR_{b}R_{c}, wherein each occurrence of Rₐ, R_{b}, and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety; and

R₃ is hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, —ORₐ, —OC(═O)Rₐ, —OC(═O)ORₐ, —OC(═O)N(Rₐ)₂, —NR_{b}R_{c}, —NHC(═O)Rₐ, —NRₐ C(═O)NR_{b}R_{c}, —NRₐ C(═O)ORₐ, —SRₐ, or —S(═O)Rₐ, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety.

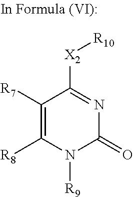

X₂ is selected from the group consisting of CR_{d}Rₑ, NR_{f}, C═O, O and S, wherein each occurrence of R_{d}, Rₑ and R_{f} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or heteroarylthio moiety;

each instance of R₇, R₈ and R₁₀ is, independently, hydrogen, alkyl, alkenyl, alkynyl, cycloalkyl, cycloalkenyl, heterocycloalkyl, heterocycloalkenyl, aryl, heteroaryl, halo, nitro, CN, —COORₐ, —C(═O)Rₐ, —C(═O)NR_{b}R_{c}, —ORₐ, —OC(═O)Rₐ, —OC(═O)ORₐ, —OC(═O)N(Rₐ)₂, —N₃, —NR_{b}R_{c}, —NHC(═O)Rₐ, —NRₐ C(═O)NR_{b}R_{c}, —NRₐ C(═O)ORₐ, —SCN, —SRₐ, —S(═O)Rₐ, —S(═O)₂Rₐ, or an amino protecting group, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety; and

R₉ is hydrogen, alkyl, alkenyl, alkynyl, cycloalkyl, cycloalkenyl, heterocycloalkyl, heterocycloalkenyl, aryl, heteroaryl, —COORₐ, —C(═O)Rₐ, —C(═O)NR_{b}R_{c}, —NR_{b}R_{c}, wherein each occurrence of Rₐ, R_{b} and R_{c} is independently a hydrogen, a protecting group, an aliphatic moiety, a heteroaliphatic moiety, an acyl moiety, an aryl moiety, a heteroaryl moiety, alkoxy, aryloxy, alkylthio, arylthio, amino, alkylamino, dialkylamino, heteroaryloxy, or a heteroarylthio moiety.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆alkyl.

The term “alkyl” refers to an straight or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉alkyl”). In some embodiments, an alkyl group has 1 to 8 carbon atoms (“C₁₋₈ alkyl”). In some embodiments, an alkyl group has 1 to 7 carbon atoms (“C₁₋₇alkyl”). In some embodiments, an alkyl group has 1 to 6 carbon atoms (“C₁₋₆alkyl”). In some embodiments, an alkyl group has 1 to 5 carbon atoms (“C₁₋₅ alkyl”). In some embodiments, an alkyl group has 1 to 4 carbon atoms (“C₁₋₄alkyl”). In some embodiments, an alkyl group has 1 to 3 carbon atoms (“C₁₋₃alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is unsubstituted C₁₋₂₀ alkyl (e.g., —CH₃). In certain embodiments, the alkyl group is substituted C₁₋₂₀ alkyl.

The term “alkylene” refers to a divalent alkyl group, i.e., an alkyl group as defined herein which is connected to the parent molecule via the removal of two or more hydrogen atoms.

The term “alkenyl” refers to a straight or branched hydrocarbon group containing one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉alkenyl”). In some embodiments, an alkenyl group has 2 to 8 carbon atoms (“C₂₋₈alkenyl”). In some embodiments, an alkenyl group has 2 to 7 carbon atoms (“C₂₋₇alkenyl”). In some embodiments, an alkenyl group has 2 to 6 carbon atoms (“C₂₋₆alkenyl”). In some embodiments, an alkenyl group has 2 to 5 carbon atoms (“C₂₋₅alkenyl”). In some embodiments, an alkenyl group has 2 to 4 carbon atoms (“C₂₋₄alkenyl”). In some embodiments, an alkenyl group has 2 to 3 carbon atoms (“C₂₋₃alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), 1,4-butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is unsubstituted C₂₋₂₀ alkenyl. In certain embodiments, the alkenyl group is substituted C₂₋₂₀ alkenyl.

The term “alkynyl” refers to a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”).

In some embodiments, an alkynyl group has 2 to 7 carbon atoms (“C₂₋₇ alkynyl”). In some embodiments, an alkynyl group has 2 to 6 carbon atoms (“C₂₋₄ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is unsubstituted C₂₋₂₀ alkynyl. In certain embodiments, the alkynyl group is substituted C₂₋₂₀ alkynyl.

The term “cycloalkyl” refers to a saturated monocyclic, bicyclic, or tricyclic hydrocarbon ring system having 3 to 10 carbon atoms (“C₃₋₁₀ cycloalkyl”) and zero heteroatoms in the ring system. “Cycloalkyl” also includes ring systems wherein the cycloalkyl ring is fused with one or more aryl or heteroaryl groups wherein the point of attachment is on the cycloalkyl ring, and in such instances, the number of carbons continue to designate the number of carbons in the cycloalkyl ring system. In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₄ cycloalkyl”). In some embodiments, a cycloalkyl group has 5 to 6 ring carbon atoms (“C₅₋₄ cycloalkyl”). In some embodiments, a cycloalkyl group has 5 to 10 ring carbon atoms (“C₅₋₁₀ cycloalkyl”). Exemplary C₃₋₄ cycloalkyl groups include, without limitation, cyclopropyl (C₃), cyclobutyl (C₄), cyclopentyl (C₅), cyclohexyl (C₆), and the like. Exemplary C₃₋₈ cycloalkyl groups include, without limitation, the aforementioned C₃₋₄ cycloalkyl groups as well as cycloheptyl (C₇), cyclooctyl (C₈), bicyclo[2.2.1]heptanyl (C₇), bicyclo[2.2.2]octanyl (C₈), and the like. Exemplary C₃₋₁₀ cycloalkyl groups include, without limitation, the aforementioned C₃₋₈ cycloalkyl groups as well as cyclononyl (C₉), cyclodecyl (C₁₀) octahydro-1H-indenyl (C₉), decahydronaphthalenyl (C₁₀), spiro[4.5]decanyl (C₁₀), adamantanyl (C₁₀), and the like. Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The term “cycloalkenyl” refers to non-aromatic monocyclic, bicyclic, or tricyclic hydrocarbon ring system having 3 to 10 carbon atoms (“C₃₋₁₀ cycloalkyl”), one or more double bonds, and zero heteroatoms in the ring system. “Cycloalkenyl” also includes ring systems wherein the cycloalkenyl ring is fused with one or more aryl or heteroaryl groups wherein the point of attachment is on the cycloalkenyl ring, and in such instances, the number of carbons continue to designate the number of carbons in the cycloalkenyl ring system. In some embodiments, a cycloalkenyl group has 3 to 8 ring carbon atoms (“C₃₋₈ cycloalkenyl”). In some embodiments, a cycloalkenyl group has 3 to 6 ring carbon atoms (“C₃₋₆ cycloalkenyl”). In some embodiments, a cycloalkenyl group has 5 to 6 ring carbon atoms (“C₅₋₆ cycloalkenyl”). In some embodiments, a cycloalkyl group has 5 to 10 ring carbon atoms (“C₅₋₁₀ cycloalkenyl”). Exemplary C₃₋₆cycloalkenyl groups include, without limitation, cyclopropenyl (C₃), cyclobutenyl (C₄), cyclopentenyl (C₅), cyclohexenyl (C₆), cyclohexadienyl (C₆), and the like. Exemplary C₃₋₈ cycloalkenyl groups include, without limitation, the aforementioned C₃₋₆ cycloalkenyl groups as well as cycloheptenyl (C₇), cycloheptadienyl (C₇), cycloheptatrienyl (C₇), cyclooctenyl (C₈), and the like. Exemplary C₃₋₁₀ cycloalkenyl groups include, without limitation, the aforementioned C₃₋₈ cycloalkenyl groups as well as cyclononenyl (C₉), cyclodecenyl (C₁₀), and the like. Unless otherwise specified, each instance of a cycloalkenyl group is independently unsubstituted (an “unsubstituted cycloalkenyl”) or substituted (a “substituted cycloalkenyl”) with one or more substituents. In certain embodiments, the cycloalkenyl group is unsubstituted C₃₋₁₀ cycloalkenyl. In certain embodiments, the cycloalkenyl group is substituted C₃₋₁₀ cycloalkenyl.

The term “heterocycloalkyl” refers to a saturated 5-8 membered monocyclic, 8-12 membered bicyclic, or 11-14 membered tricyclic ring system (collectively referred to as a “3-14 membered heterocycloalkyl”) having ring carbon atoms and one to four heteroatoms ring heteroatoms, wherein each heteroatom is independently selected from the group consisting of oxygen, nitrogen, sulfur, phosphorus, boron, silicon, and selenium. In heterocycloalkyl groups that contain one or more nitrogen atoms, the point of attachment can be a carbon or nitrogen atom, as valency permits. A heterocycloalkyl group can either be monocyclic (“monocyclic heterocycloalkyl”) or a fused, bridged or spiro ring system such as a bicyclic system (“bicyclic heterocycloalkyl”). Heterocycloalkyl bicyclic ring systems can include one or more heteroatoms in one or both rings. “Heterocycloalkyl” also includes ring systems wherein the heterocycloalkyl ring, as defined above, is fused with one or more cycloalkyl or cycloalkenyl groups wherein the point of attachment is either on either ring, or ring systems wherein the heterocycloalkyl ring, as defined above, is fused with one or more aryl or heteroaryl groups, wherein the point of attachment is on the heterocycloalkyl ring, and in such instances, the number of ring members continue to designate the number of ring members in the heterocycloalkyl ring system. Unless otherwise specified, each instance of heterocycloalkyl is independently unsubstituted (an “unsubstituted heterocycloalkyl”) or substituted (a “substituted heterocycloalkyl”) with one or more substituents. In certain embodiments, the heterocycloalkyl group is unsubstituted 5-10 membered heterocycloalkyl. In certain embodiments, the heterocycloalkyl group is substituted 5-10 membered heterocycloalkyl.

In some embodiments, a heterocycloalkyl group is a 5-10 membered ring system having ring carbon atoms and 1-4 ring heteroatoms (“5-10 membered heterocycloalkyl”). In some embodiments, a heterocycloalkyl group is a 5-8 membered ring system having ring carbon atoms and 1-4 ring heteroatoms (“5-8 membered heterocycloalkyl”). In some embodiments, a heterocycloalkyl group is a 5-6 membered ring system having ring carbon atoms and 1-4 ring heteroatoms (“5-6 membered heterocycloalkyl”). In some embodiments, the 5-6 membered heterocycloalkyl has 1-3 ring heteroatoms. In some embodiments, the 5-6 membered heterocycloalkyl has 1-2 ring heteroatoms. In some embodiments, the 5-6 membered heterocycloalkyl has one ring heteroatom.

Exemplary 5-membered heterocycloalkyl groups containing one heteroatom include, without limitation, tetrahydrofuranyl, dihydrofuranyl, tetrahydrothiophenyl, and pyrrolidinyl. Exemplary 5-membered heterocycloalkyl groups containing two heteroatoms include, without limitation, dioxolanyl, oxasulfuranyl, and disulfuranyl. Exemplary 5-membered heterocycloalkyl groups containing three heteroatoms include, without limitation, triazolinyl, oxadiazolinyl, and thiadiazolinyl. Exemplary 6-membered heterocycloalkyl groups containing one heteroatom include, without limitation, piperidinyl, tetrahydropyranyl, and thianyl. Exemplary 6-membered heterocycloalkyl groups containing two heteroatoms include, without limitation, piperazinyl, morpholinyl, dithianyl, and dioxanyl. Exemplary 7-membered heterocycloalkyl groups containing one heteroatom include, without limitation, azepanyl, oxepanyl, and thiepanyl. Exemplary 8-membered heterocycloalkyl groups containing one heteroatom include, without limitation, azocanyl, oxecanyl, and thiocanyl.

The term “heterocycloalkenyl” refers to a nonaromatic 5-8 membered monocyclic, 8-12 membered bicyclic, or 11-14 membered tricyclic ring system (collectively referred to as a “3-14 membered heterocycloalkenyl”) having ring carbon atoms and one to four heteroatoms ring heteroatoms, wherein each heteroatom is independently selected from the group consisting of oxygen, nitrogen, sulfur, phosphorus, boron, silicon, and selenium. In heterocycloalkenyl groups that contain one or more nitrogen atoms, the point of attachment can be a carbon or nitrogen atom, as valency permits. A heterocycloalkenyl group can either be monocyclic (“monocyclic heterocycloalkenyl”) or a fused, bridged or spiro ring system such as a bicyclic system (“bicyclic heterocycloalkenyl”). Heterocycloalkenyl bicyclic ring systems can include one or more heteroatoms in one or both rings. “Heterocycloalkenyl” also includes ring systems wherein the heterocycloalkenyl ring, as defined above, is fused with one or more cycloalkyl or cycloalkenyl groups wherein the point of attachment is either on either ring, or ring systems wherein the heterocycloalkenyl ring, as defined above, is fused with one or more aryl or heteroaryl groups, wherein the point of attachment is on the heterocycloalkenyl ring, and in such instances, the number of ring members continue to designate the number of ring members in the heterocycloalkenyl ring system. Unless otherwise specified, each instance of heterocycloalkenyl is independently unsubstituted (an “unsubstituted heterocycloalkenyl”) or substituted (a “substituted heterocycloalkenyl”) with one or more substituents. In certain embodiments, the heterocycloalkenyl group is unsubstituted 5-10 membered heterocycloalkenyl. In certain embodiments, the heterocycloalkenyl group is substituted 5-10 membered heterocycloalkenyl.

In some embodiments, a heterocycloalkenyl group is a 5-10 membered ring system having ring carbon atoms and 1-4 ring heteroatoms (“5-10 membered heterocycloalkenyl”). In some embodiments, a heterocycloalkenyl group is a 5-8 membered ring system having ring carbon atoms and 1-4 ring heteroatoms (“5-8 membered heterocycloalkenyl”). In some embodiments, a heterocycloalkenyl group is a 5-6 membered ring system having ring carbon atoms and 1-4 ring heteroatoms (“5-6 membered heterocycloalkenyl”). In some embodiments, the 5-6 membered heterocycloalkenyl has 1-3 ring heteroatoms. In some embodiments, the 5-6 membered heterocycloalkenyl has 1-2 ring heteroatoms. In some embodiments, the 5-6 membered heterocycloalkenyl has one ring heteroatom.

Exemplary 5-membered heterocycloalkenyl groups containing one heteroatom include, without limitation, dihydrothiophenyl, dihydropyrrolyl, 3,4-dihydropyrrol-2-one, and pyrrolyl-2,5-dione. Exemplary 6-membered heterocycloalkenyl groups containing one heteroatom include, without limitation, dihydropyridinyl. Exemplary bicyclic heterocycloalkenyl groups include, without limitation, indolinyl, isoindolinyl, dihydrobenzofuranyl, dihydrobenzothienyl, tetrahydroquinolinyl, tetrahydroisoquinolinyl, decahydroquinolinyl, and decahydroisoquinolinyl, and the like.

The term “aryl” refers to a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

The term “heteroaryl” refers to an aromatic 5-8 membered monocyclic or polycyclic (i.e., 8-12 membered bicyclic, or 11-14 membered tricyclic) ring system (collectively referred to as a “5-14 membered heteroaryl”) having ring carbon atoms and one to four heteroatoms ring heteroatoms, wherein each heteroatom is independently selected from the group consisting of oxygen, nitrogen, sulfur, phosphorus, boron, silicon, and selenium. In heteroaryl groups that contain one or more nitrogen atoms, the point of attachment can be a carbon or nitrogen atom, as valency permits. Heteroaryl bicyclic or tricyclic ring systems can include one or more heteroatoms in one more of the rings. “Heteroaryl” includes ring systems wherein the heteroaryl ring, as defined above, is fused with one or more cycloalkyl, cycloalkenyl, heterocycloalkyl, or heterocycloalkenyl, wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system (“5-10 membered heteroaryl”). In some embodiments, a heteroaryl group is a 5-8 membered aromatic ring system having ring carbon atoms and 1-4 ring heteroatoms provided in the aromatic ring system (“5-8 membered heteroaryl”). In some embodiments, a heteroaryl group is a 5-6 membered aromatic ring system having ring carbon atoms and 1-4 ring heteroatoms provided in the aromatic ring system (“5-6 membered heteroaryl”). In some embodiments, the 5-6 membered heteroaryl has 1-3 ring heteroatoms. In some embodiments, the 5-6 membered heteroaryl has 1-2 ring heteroatoms. In some embodiments, the 5-6 membered heteroaryl has 1 ring heteroatom. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, purinyl, [1,2,3]triazolo[4,5-b]pyrazinyl, [1,2,5]thiadiazolo[3,4-b]pyrazinyl, and [1,2,5]oxadiazolo[3,4-b]pyrazinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of “alkyl” and refers to an optionally substituted alkyl group, as defined herein, substituted by an optionally substituted heteroaryl group, as defined herein, wherein the point of attachment is on the alkyl moiety.

Heteroarylalkenyl” is a subset of “alkenyl” and refers to an optionally substituted alkenyl group, as defined herein, substituted by an optionally substituted heteroaryl group, as defined herein, wherein the point of attachment is on the alkenyl moiety.

Alkyl, alkenyl, alkynyl, cycloalkyl, cycloalkenyl, heteroalkyl, heteroalkenyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” cycloalkyl, “substituted” or “unsubstituted” cycloalkenyl, “substituted” or “unsubstituted” heteroalkyl, “substituted” or “unsubstituted” heteroalkenyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Alkyl, alkenyl, alkynyl, cycloalkyl, heterocycloalkyl, cycloalkenyl, heterocycloalkenyl, aryl, and heteroaryl mentioned above include both substituted and unsubstituted moieties. Exemplary substituents on cycloalkyl, heterocycloalkyl, cycloalkenyl, heterocycloalkenyl, aryl, and heteroaryl include, but are not limited to, alkyl, alkenyl, alkynyl, cycloalkyl, cycloalkenyl, heterocycloalkyl, heterocycloalkenyl, aryl, heteroaryl, alkoxy, aryloxy, heteroaryloxy, amino (—NH₂), alkylamino, arylamino, heteroarylamino, hydroxy (—OH), halo (—F, —Br, —I, —Cl), oxo (O═), thioxo (S═), thio (—SH), silyl, thioacyl, acylthio, alkylthio, arylthio, alkylsulfonyl, arylsulfonyl, heteroarylsulfonyl, aminoacyl, aminothioacyl, amidino, amido, thioureido, thiocyanato (—SCN), sulfonamide, guanidino, ureido, cyano (—CN), nitro (—NO₂), acyl, thioacyl, acyloxy, carbamido, carbamyl, carboxylic acid (—COOH), and carboxylic ester. Exemplary substituents on alkyl, alkenyl, or alkynyl include all of the above-recited substituents except alkyl, alkenyl, or alkynyl.

The term “alkoxy” or “alkyloxy” refers to an —O-alkyl radical, wherein alkyl is optionally substituted alkyl as defined herein. Examples of alkoxy include, but are not limited to, methoxy, ethoxy, n-propoxy, isopropoxy, n-butoxy, iso-butoxy, sec-butoxy, and tert-butoxy.

The term “aryloxy” refers to an —O-aryl, wherein aryl is optionally substituted aryl as defined herein.

The term “heteroaryloxy” refers to an —O-heteroaryl, wherein heteroaryl is optionally substituted heteroaryl as defined herein.

The term “acyl” refers to an —C(═O)R radical in which R is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “acyloxy” refers to an —OC(═O)R radical in which R is selected from the group consisting of optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “alkylthio” refers to an —S-alkyl radical, wherein alkyl is optionally substituted alkyl as defined herein. Examples of alkoxy include, but are not limited to, methoxy, ethoxy, n-propoxy, isopropoxy, n-butoxy, iso-butoxy, sec-butoxy, and tert-butoxy.

The term “arylthio” refers to an —S-aryl, wherein aryl is optionally substituted aryl as defined herein.

The term “heteroarylthio” refers to an —S-heteroaryl, wherein heteroaryl is optionally substituted heteroaryl as defined herein.

The term “acylthio” refers to an —SC(═O)R radical in which R is selected from the group consisting of optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “thioacyl” refers to an —C(═O)SR radical in which R is selected from the group consisting of optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “amino” refers to —NH₂, alkylamino, or arylamino.

The term “alkylamino” refers to the group —N(R)-alkyl, in which alkyl is optionally substituted alkyl, as defined herein, and each instance of R is independently selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “arylamino” refers to an —N(R)-aryl, in which aryl is optionally substituted aryl, as defined herein, and each instance of R is independently selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “heteroarylamino” refers to an —N(R)-heteroaryl, in which heteroaryl is optionally substituted heteroaryl, as defined herein, and each instance of R is independently selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “amido” or “amino acyl” refers to —NRC(═O)R′ in which each of R and R′, independently, is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “aminothioacyl” refers to —NRC(═S)R′ in which each instance of R and R′, independently, is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “amidino” refers to —NRC(═NR)R′ or —C(═NR)NRR′ in which each instance of R and R′, independently, is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “carbamido” or “acylamino” refers to —C(═O)NRR′ in which each of R and R′, independently, is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl. The term “carbamyl” is a subset of carbamido, and refers to the group —C(O)NH₂, i.e., wherein R and R′ are both hydrogen.

The term “ureido” refers to —NRC(═O)NRR′ in which each of R and R′, independently, is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “thioureido” refers to —NRC(═S)NRR′ in which each of R and R′, independently, is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “guanidino” refers to —NRC(═NR)NRR′ in which each of R and R′, independently, is selected from the group consisting of hydrogen, optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “silyl” refers to a group —SiR₃ in which each of R independently is selected from the group consisting of optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl

The term “alkylsulfonyl” refers to the group —SO₂-alkyl, wherein alkyl is optionally substituted alkyl as defined herein.

The term “arylsulfonyl” refers to the group —SO₂-aryl, wherein aryl is optionally substituted aryl as defined herein.

The term “heteroarylsulfonyl” refers to the group —SO₂-heteroaryl, wherein heteroaryl is optionally substituted heteroaryl as defined herein.

The term “sulfonamide” or “sulfonamido” refers to the group —SO₂NRR′, —SO₂NHR′, —SO₂NH₂, —NHSO₂R′, or —NRSO₂R′ in which each of R and R′, independently, is selected from the group consisting of optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

The term “carboxylic ester” refers to —CO₂R in which R is selected from the group consisting of optionally substituted alkyl, optionally substituted alkenyl, optionally substituted alkynyl, optionally substituted cycloalkyl, optionally substituted cycloalkenyl, optionally substituted heterocycloalkyl, optionally substituted heterocycloalkenyl, optionally substituted aryl, and optionally substituted heteroaryl.

Nitrogen and oxygen protecting groups (also respectively referred to as amino and hydroxyl protecting groups) are known in the art and include those described in detail in Protecting Groups in Organic Synthesis, T. W. Greene and P. G. M. Wuts, 3rd edition, John Wiley & Sons, 1999, incorporated herein by reference.

Nitrogen atoms may be protected in a variety of ways, for example, as amides, carbamates, sulfonamides, and the like. Exemplary amide nitrogen protecting groups include but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide. Exemplary carbamate nitrogen protecting groups include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate. Exemplary sulfonamide nitrogen protecting groups include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′ dimethoxynaphthylmethyl)benzenesulfonamide (DNMBS), benzylsulfonamide, trifluoromethylsulfonamide, and phenacylsulfonamide. Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

These and other exemplary substituents are described in more detail in the Detailed Description, Examples, and claims. The invention is not intended to be limited in any manner by the above exemplary listing of substituents.

“Salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

Any of the compounds described herein can be prepared by conventional chemical transformations (including protecting group methodologies), e.g., those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof. The compounds can also be synthesized in manners similar to those described, e.g., Repub. Korean Kongkae Taeho Kongbo (2009), KR 2009033583 A 20090406; PCT Int. Appl. (2008), WO 2008025694; PCT Int. Appl. (1998), WO 2007038452; U.S. Pat. No. 5,968,965; PCT Int. Appl. No. WO 9822433; PCT Int. Appl. (1998), WO 9822432; PCT Int. Appl. (1997), WO 9727852; Jpn. Kokai Tokkyo Koho (1994), JP Patent No. 06271762; JP Patent No. 06263969; JP Patent No. 06234890; U.S. Pat. No. 3,912,492; U.S. Pat. No. 3,812,121 with necessary modifications as recognized by those skilled in the art.

A compound thus synthesized can be further purified by flash column chromatography, high performance liquid chromatography, crystallization, or any other suitable methods. The compounds mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Alternatively, certain compounds as described herein, e.g., Compounds 1, 2, 3 and 4, can be purchased from a commercial ventor, e.g., Molport (Riga, Latvia), AMRI (Budapest, Hungary), Enamine (Kiev, Ukraine) and Life Chemicals (Kiev, Ukraine), respectively.

### (ii) Peptides

The peptides disclosed herein that are capable of disrupting formation of NP trimers and thus inhibiting viral replication comprise a fragment of an influenza viral NP (“NP fragment”) that encompasses an Arg residue corresponding to R416 in SEQ ID NO:1, or a functional equivalent thereof, which can share at least 80% sequence identity (e.g., at least 85%, 90%, 95%, or 98%) with the NP fragment and possessing anti-influenza viral activity.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In one example, the NP fragment includes the tail-loop region in an NP protein, e.g., residues 402 to 428 in SEQ ID NO:1 or a corresponding fragment thereof, which can be determined by comparing the sequence of an NP protein of interest with SEQ ID NO:1. In another example, the NP fragment, or a portion thereof, corresponds to the segment spanning from the residues 409 to 418 in SEQ ID NO:1 or residues 411 to N417 in SEQ ID NO:1.

Functional equivalents of the NP fragment can be prepared by introducing substitutions, deletions and/or insertions into an NP fragment. In some examples, a functional equivalent contains at most 3, preferably at most 2, more preferably at most 1 mutation as compared to its wild-type counterpart.

The peptides described herein can be designed based on the amino acid sequences of influenza viral NPs, which are well known in the art with SEQ ID NO:1 as a representative species. For example, sequences of such viral NPs can be obtained via routine technology, e.g., retrieved from GenBank using SEQ ID NO:1 as a query. Such peptides can consist of the NP fragments/functional equivalents described above, or contain additional amino acids at either end of the NP fragments/functional equivalents. Preferably, the peptides have lengths less than 100 amino acids, e.g., 6-100, 6-50, 6-30, or 8-20 amino acids.

The peptides can be linear or cyclic. Methods for making cyclic peptides are well known in the art. In one example, two cysteine residues can be incorporated into the peptides in regions flanking the NP fragments/functional equivalents. A disulfide bond can be formed between the two cysteine residues to form a cyclic peptide. Examples of such peptides include, but are not limited to, CTFSVQRNC (SEQ ID NO:2), CPTFSVQRNLC (SEQ ID NO:3), or CQPTFSVQRNLC (SEQ ID NO:4). Alternatively, the peptides can contain modified amino acid residues to improve in vivo stability following routine technology known in the art.

The peptides described herein can be made by any conventional methods, i.e., recombinant technology or standard methods of solid phase peptide chemistry well known to any one of ordinary skill in the art. For example, the peptides may be synthesized by solid phase chemistry techniques following the procedures described by Steward et al. in Solid Phase Peptide Synthesis, 2nd Ed., Pierce Chemical Company, Rockford, Ill., (1984) using a Rainin PTI Symphony synthesizer. For solid phase peptide synthesis, techniques may be found in Stewart et al. in “Solid Phase Peptide Synthesis”, W. H. Freeman Co. (San Francisco), 1963 and Meienhofer, Hormonal Proteins and Peptides, 1973, 2 46. For classical solution synthesis, see for example Schroder et al. in “The Peptides”, volume 1, Acacemic Press (New York). In general, such methods comprise the sequential addition of one or more amino acids or suitably protected amino acids to a growing peptide chain on a polymer. Normally, either the amino or carboxyl group of the first amino acid is protected by a suitable protecting group. The protected and/or derivatized amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth.

### III. Pharmaceutical Compositions Comprising Anti-Influenza Virus Agents and Uses Thereof in Inhibiting Viral Replication and Treating Viral Infection

Agents that inhibit viral replication via, e.g., disrupting the salt bridge in a viral NP protein corresponding to the E339 . . . R416 salt bridge in SEQ ID NO:1, can be used for inhibiting influenza A viral replication and/or treating influenza viral infection. Such agents include any of the compounds/peptides disclosed herein, or an agent identified in the screening methods also disclosed herein.

Any of the anti-flu agents described herein can be mixed with a pharmaceutically acceptable carrier or excipient to form a pharmaceutical composition for use in inhibiting influenza viral replication and/or treating infection caused by an influenza virus. As used herein, “inhibiting,” “inhibition,” “inhibit,” “inhibitor,” and the like, refer to the ability of an anti-flu agent to reduce, slow, halt, or prevent activity of a particular biological process (e.g., influenza virus replication) in a cell relative to a control vehicle. In some instances, an anti-flu agent can inhibit the level of viral replication by at least 20% (e.g., 30%, 40%, 50%, 60%, 70%, 80%, 90%, or 95%).

The term “treating” or “treatment” refers to administering one or more anti-influenza A virus agent (e.g., the compounds and peptides described herein) to a subject (e.g., a human patient), who has influenza virus infection, a symptom of or a predisposition toward it, with the purpose to confer a therapeutic effect, e.g., to cure, relieve, alter, affect, ameliorate, or prevent the infection, the symptom of or the predisposition toward it. Such a subject can be identified by a health care professional based on results from any suitable diagnostic method.

The carrier in the pharmaceutical composition must be “acceptable” in the sense of being compatible with the active ingredient of the formulation (and preferably, capable of stabilizing it) and not deleterious to the subject to be treated. For example, solubilizing agents such as cyclodextrins, which form more soluble complexes with the anti-viral agents described herein, or more solubilizing agents, can be utilized as pharmaceutical carriers for delivery of the anti-viral agents. Examples of other carriers include colloidal silicon dioxide, magnesium stearate, sodium lauryl sulfate, and D&C Yellow #10. See, e.g., Remington: The Science and Practice of Pharmacy, 21st Edition (Lippincott Williams & Wilkins, 2005); and Goodman and Gilman's “The Pharmacological Basis of Therapeutics”, Tenth Edition, Gilman, J. Hardman and L. Limbird, eds., McGraw-Hill Press, 155-173, 2001.

Pharmaceutically acceptable excipients/carriers include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and mixtures thereof.

To practice the treatment method described herein, one can contact cells infected with an influenza A virus with any of the pharmaceutical compositions described herein, comprising an effective amount of an anti-flu compound or peptide as also described herein. In some embodiments, this is performed by administering the pharmaceutical composition to a subject in need of the treatment. In other embodiments, the method described herein is carried out in vitro.

An “effective amount” is the amount of the anti-flu agent, either alone, or together with further doses, that produces one or more desired responses, e.g. inhibit viral replication. In the case of treating an infection caused by an influenza virus, the desired responses include inhibiting the progression of the disease or alleviating one or more symptoms associated with influenza infection. This may involve only slowing the progression of the disease temporarily, although more preferably, it involves halting the progression of the disease permanently. This can be monitored by routine methods. The desired responses to treatment of the disease or condition also can be delaying the onset or even preventing the onset of the disease or condition.

Effective amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some embodiments, the amount of an anti-flu agent as described herein is effective in inhibiting viral infection. In other embodiments, the amount of an anti-flu agent is effective in alleviating one or symptoms associated with influenza viral infection.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or other non-human animals, for example mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In certain embodiments, the non-human animal is a mammal. The non-human animal may be a male or female and at any stage of development. A non-human animal may be a transgenic animal.

In some examples, the subject in need of the treatment described herein can be a human patient has or is suspected of having infection with influenza virus, e.g., a wild-type influenza A virus (e.g., H1N1, H5N1, or H3N2) or with a mutant influenza virus, such as one that has a mutated NP protein, e.g., Y289H, Y52H, or Y52H/Y289H. A subject suspected of having infection caused by an influenza virus might show one or more symptoms of the infection, e.g., fever, cough, nasal congestion, body aches, fatigue, headache, watering eyes, diarrhea and/or abdominal pain.

Any of the pharmaceutical compositions described herein can be administered to a subject in need of the treatment via any conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the anti-flu agent described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

A composition for oral administration can be any orally acceptable solid dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as can be required. Additionally, the present invention contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

A pharmaceutical composition of the invention can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered. by rapid inhalation through the nasal passage from a container of the powder held close to the nares.

A nasal aerosol or inhalation composition can be prepared according to techniques well known in the art of pharmaceutical formulation. Formulations for nasal administration may, for example, comprise from about as little as 0.1% (w/w) and as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

In certain embodiments, an effective amount of an anti-flu agent as described herein for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In other embodiments, the anti-flu agent may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. The compounds or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Also encompassed by the present disclosure are kits (e.g., pharmaceutical packs). The kits provided may comprise an inventive pharmaceutical composition or compound/peptide and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety for the purposes or subject matter referenced herein.

### EXAMPLES

### Materials and Methods

### Viruses and Cells

HEK293T and MDCK cells were grown in Dulbecco's Modified Eagle Medium (DMEM) containing 10% fetal bovine serum (FBS). Cells were maintained at 37° C. and 5% CO₂. The NP WT and mutant MDCK stable cell lines were established by the Retro-X™ Universal Packaging System (Clontech Laboratories Inc). The system includes the GP2-293 cell line, which has the viral gag and pol genes incorporated in its genome. Influenza virus A/WSN/33 (H1N1) was propagated in MDCK cells and embryonated in chicken eggs. The virus titer was determined by plaque assays.

### Plasmids

Plasmids pcDNA-PB1, -PB2, -PA, -NP, pClneo-NP-HA and -NP-Flag, encoding, respectively, the PB1, PB2, PA and NP proteins of the WSN virus have been described previously (Fodor E, et al. (2002) Journal of Virology 76:8989-9001). NP Δ402-428, R416A, E339A and E339A/Δ402-428 were generated by QuickChange Site-Directed Mutagenesis Kit (Stratagene). The DNA sequences of desired mutations were confirmed by sequencing. Plasmid pEGFP-tail-loop was constructed with the WSN virus tail-loop sequence (amino acids 402-428) inserted into pEGFP-C3 vector (BD Biosciences Clontech). The pPOLI-Luc-RT plasmid contained the firefly luciferase open reading frame in negative orientation flanked by the non-coding regions of non-structural (NS) sequence of the WSN virus and by the human RNA polymerase I promoter and the mouse RNA polymerase I terminator (Neumann G, et al. (1994) Virology, 202:477-479). The Retro-X™ Universal Packaging System contains the pLNCX and pVSV-G plasmids. Plasmid pLNCX is designed for retrovial gene delivery and expression. Plasmid pVSV-G can express pantropic vesicular stomatitis virus envelope glycoproteins (VSV-G). The pLNCX-NP-V5 plasmid contains NP gene which was inserted into the pLNCX vector and the V5 tag is in the C-terminal.

### Expression and Purification of NP and Mutants

The NP gene from the WSN virus coding a 498-residue protein was cloned into vector pET15b and expressed in BL21-CodonPlus® (DE3)-RIPL cell (Stratagene). The purification of NP and its mutants for AUC analyses followed a previously reported procedure (Ye Q, et al. (2006) Nature 444:1078-1082). In brief, the expressed NP proteins in E. coli were purified using Chelating Sepharose FF, Hiprep heparin FF and HiLoad Superdex 200 16×60 columns (GE Healthcare). The purification buffer contains 50 mM Tris, pH 8.0, 200 mM NaCl, 2 mM EDTA, and 2 mM β-mercaptoethanol. The NP obtained at this stage contained endogenous RNA derived from E. coli. In order to obtain the NP protein free of RNA, the purified proteins were incubated with ribonuclease A. Purity of the NP proteins thus prepared were confirmed by SDS-PAGE and MS analysis.

### Luciferase-Based Reporter Assays

Approximately 4×10⁵ HEK293T cells in 6-well plates were transfected with pPOLI-Luc-RT, pcDNA-PB1, -PB2, -PA and -NP (1 μg each) by jetPEI transfection reagent (PolyPlus). At 30 h after transfection, the cell extracts were examined for firefly luciferase levels with luciferase assay system (Promega) and measured by luminescence reader.

### Western Blot Analysis

NP WT and mutant MDCK stable cells with expressed NP protein or HEK 293T cells were transfected with 2 μg plasmids. After 24 h, the cells were incubated with DMEM containing 0.2% bovine serum albumin (BSA) and 25 mM HEPES, and infected with influenza virus A/WSN/33 at a multiplicity of infection (MOI) of 0.2 for 9 h. Cells were collected and lysed with 50 μl of lysis buffer (0.2% Triton X-100 in PBS). The supernatant was added with the SDS sample buffer. Aliquots of the supernatant were fractionated on a 10% NuPAGE Novex Bis-Tris gel (Invitrogen) and electro-blotted onto BioTrace PVDF polyvinylidene fluoride transfer membrane (Pall Corporation). Membranes were blocked with skim milk for 1 h at room temperature, then incubated with specific antibodies and visualized by a Western Lightening chemiluminescence reagent plus kit (PerkinElmer Life Science).

### Plaque Assay

NP WT and mutant MDCK stable cells with expressed NP protein or HEK 293T cells were transfected with 2 μg plasmid. After 24 h, the cells were incubated with medium A (DMEM containing 0.2% BSA and 25 mM HEPES) and 25 mM HEPES and were infected with influenza virus A/WSN/33 at a MOI of 0.2 for 9 h. The cell culture supernatant was collected. Monolayer of 10⁶ MDCK cells in 6-well plates was inoculated with 1 ml of virus dilution. Serial (powers of ten) dilutions were made from cell culture supernatant. After 1 h, the inoculums were removed and the cells were washed twice with PBS. The cells were covered with 2 ml of agar medium (100 ml of 2× medium A and 100 ml of 2% agar). After 3 days, cells were fixed with 10% formaldehyde in PBS for 30 min. The agar was cleaned and 1% crystal violet in 20% ethanol was added to facilitate plaque counting.

### Analytical Ultracentrifuge Analysis

Sedimentation velocity (SV) experiment was performed by a Beckman-Coulter XL-I analytical ultracentrifuge (Fullerton, Calif., USA). Samples and buffers were loaded into 12-mm standard double-sector Epon charcoal-filled centrepieces and mounted in an An-60 Ti rotor. SV experiments were performed at rotor speed of 40,000 rpm at 20° C. The signals of samples were monitored at 280 nm. The partial specific volume of influenza A NP is 0.7256. The raw experimental data were analyzed by Sedfit (http://www.analyticalultracentrifugation.com/default.htm) and the plots were generated by MATLAB (MathWork, Inc.). The calculated c(s, fr) distribution was shown in two dimensions with grid lines representing the s and fr grids in the thermograph. Below this c(s, fr) surface a contour plot of the distribution was projected into the s-fr plane, where the magnitude of c(s, fr) was indicated by contour lines at constant c(s, fr) in equidistant intervals of c. Contour plots were transformed from the calculated c(s, fr) distribution and were shown as c(s, M) distributions. The dotted lines indicate lines of fᵣ (frictional ratio). The signal of the c(s,M) distribution is indicated by the color temperature. The insert grayscale bars in the right panels indicate the residuals bitmap of each fit. All samples were visually checked for clarity after ultracentrifugation, and no precipitation was observed.

Differential distribution of sedimentation coefficients and fictional ratios c(s, fr) were calculated with sedfit using c(s,*) model with Equation 1 (Brown P H, et al. (2006) Biophysical Journal 90:4651-4661)

a(r,t)=∫∫c(s,fr)×(s,D(s,fr),r,t)dsdfr [Equation 1]

The c(s, fr) distribution could be transformed to a molar mass distribution for each s-value, called c(s, M) distribution, by Equation 2 (Brown P H, et al. (2006) Biophysical Journal 90:4651-4661).

a(r,t)=∫∫c(s,M)×(s,D(s,M)r,t)dsdM [Equation 2]

### Circular Dichroism Analysis

Monitoring the CD spectrum of the protein was monitored at 25° C. in a Jasco J-815 spectropolarimeter under constant N₂ flush and using a 0.1 nm path length to cell analyze the secondary structure of the enzyme. Ten repetitive scans between 250 and 180 nm were averaged. For direct comparison, all enzyme solutions were adjusted to the same protein concentration (1.0 mg ml⁻¹). Mean residue ellipticity (Φ) was obtained by the following equation:

Φ=[Φ]₂₂₂M_{MRW}/10dc

in which M_{MRW} of NP is 112.9, the mean amino acid residue weight. d is the cell path in cm, and c is the concentration of NP in mg ml⁻¹.

### Virtual Screening

All computational work was done by using Accelrys Discovery Studio™, version 2.5, and Pipeline Pilot™, version 7.5 (Accelrys, USA). The molecular structure of nucleoprotein NP from the influenza A virus was obtained from RCSB Protein Data Bank (PDB code 21QH); hydrogen atoms were added before refinement. The structures of 1.7 million compounds (the collection of the Genomics Research Center) were generated and energy optimized. The process of screening was conducted in five steps: first, pharmacophore features were developed by Phase™ based on the tail-loop and the pocket of NP structure (Ye Q, et al. (2006) Nature 444:1078-1082), and partitioned into five sub-pockets. Second, using interaction generation protocol of Discovery Studio™, we obtained interaction features, which were clustered by type. A structure-based pharmacophore model, which contained 3 hydrogen-bond donor, 1 hydrogen-bond acceptor and 1 hydrophobic, was created by using only the center of each clustered feature. Third, a modified-Lipinski filter (HBA count≦2; HBD count≦3; molecular weight≦500; AlogP≦5) based on the pharmacophore model was applied to pre-screening the 1.7 million for the sake of time-cost. Fourth, the resulted 200,000 compounds were grouped by structure similarity (Tanimoto>0.8) and the centers of each group (about 2 thousands) were subjected to energy minimization (CHARMm force field, GBMV implicit solvent model, and a maximum of 2,000 iterations) and conformational generation (a maximum number of 255 of conformation generation) for binding evaluation. Finally, pharmacophore mapping and clustering were used to find compounds that mapped to the model and that showed structural similarity to the experimental hits from the high-throughput screening assays we performed previously (Su C Y, et al. (2010) Proc Natl Acad Sci USA 107:19151-19156). There were 1,050 possible ligands that mapped to the model, which fall into five clusters structurally. Representative 24 compounds from the clusters containing experimental hits were selected for further antiviral assay in this work.

### Primer Extension Assay

MDCK stable cells were infected with the WSN virus (MOI=2) and then harvested 6 h post infection. Total RNA was extracted by the TRIzol reagent (Invitrogen). RNA samples were mixed with each ³²P-labeled primer and denatured at 95° C. for 5 min. The mixture was cooled on ice and then incubated at 45° C. for 10 min, and added with the reverse transcription buffer and enzyme (Toyobo Life Science) to start the reverse transcription reaction. Two Neuraminidase (NA) gene-specific primers (Vreede F T, et al. (2007) J Virol 81:2196-2204) and one canis 16S ribosomal RNA primer were used: NA_negative: 5′-TGGACTAGTGGGAGCA TCAT-3′ (SEQ ID NO:5; to detect vRNA, 122 nt), NA_positive: 5′-TCCAGTATGGTTTTGA TTTCCG-3′ (SEQ ID NO:6; to detect mRNA, >161 nt, and cRNA, 161 nt) and canis 16S_{—}118-99: 5′-TACTATCTCTATCGCTCCAA-3′ (SEQ ID NO:7; to detect canis rRNA, 118 nt). The reaction was stopped by addition of 8 μl 90% formamide and heating at 99° C. for min, and analyzed on 6% polyacrylamide gels containing 7 M urea in TBE (Tris-borate-EDTA buffer). Transcription products were detected by autoradiography.

### In Vitro Transcription

Approximately 10⁷ plaque forming units (pfu) of the WSN virus were incubated in a buffer containing 100 mM Tris-HCl (pH 8.0), 5 mM MgCl₂, 100 mM KCl, 1 mM DTT, 0.25% Triton N-101 and 0-4 mM peptides or compounds at 25° C. After 1 hr, the samples were incubated with 200 μM ApG, RNase-inhibitor 1 U/μl, 100 μM ATP, 50 μM CTP, 50 μM UTP, 1 μM GTP, 5 μCi [3H]UTP for 30 min at 30° C. The RNA synthesized was precipitated by 10% trichloroacetic acid (TCA) on ice for 1 hr. Glass microfiber filters (GF/C) were rinsed with 10% TCA and placed onto the vacuum filter device. The samples were spotted on the labeled GF/C filters, washed with 10% TCA and dried with 95% ethanol for 1 min, and then were counted in a scintillation counter.

### Preparation of Isogenic Recombinant Influenza Viruses

The isogenic recombinant influenza viruses were prepared as described in our recent report (Su C Y, et al. (2010) Proc Natl Acad Sci USA 107:19151-19156). The recombinant influenza pairs isogenic at the 52nd, 289th or 52nd/289th amino acid of NP was produced.

### Antiviral Assay

In a 96-well plate, 1×10⁴ MDCK cells were seeded per well and incubated for 24 h at 37° C. The MDCK cells were then incubated with the compound, then inoculated with medium alone or the WSN virus (MOI of 0.001) for 48 h at 35° C. The number of metabolically viable cells was determined by the MTS assay (Promega) or Cell-Titer Glo® (Promega).

### Cytotoxicity Assay

In a 96-well plate, 1×10⁴ MDCK cells were seeded per well and incubated for 24 h at 37° C. The MDCK cells were then incubated with compounds for 48 h at 35° C. The number of metabolically viable cells was determined by the MTS assay (Promega).

### Indirect Immunofluorescence Staining and Confocal Microscopy

MDCK cells were grown on coverslips and then were infected with the WSN virus (MOI=1) and incubated with compounds at 6 hr post infection. After 8 h post infection, cells were fixed, and immunostained with anti-NP (mouse), and then with anti-mouse conjugated fluorescein isothiocyanate (FITC) and with 4′,6-diamidino-2-phenylindole (DAPI). Immunofluorescence images were obtained by using a Leica TCS-SP2 laser scanning confocal microscope (Leica Microsystems GmbH).

### Results

### (I) Identification of the E339 . . . R416 Salt Bridge of NP as a Target in Anti-Influenza Therapy

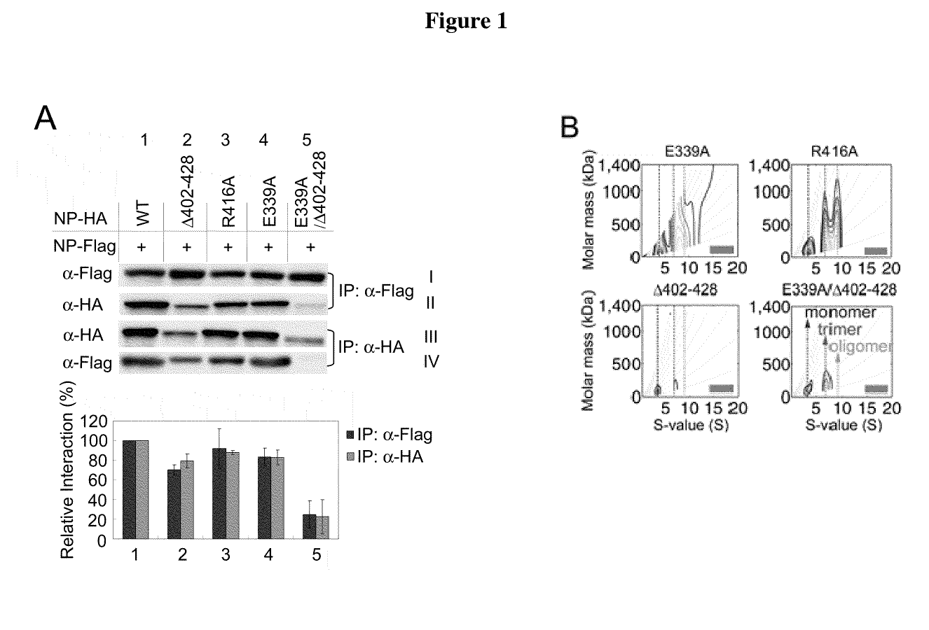

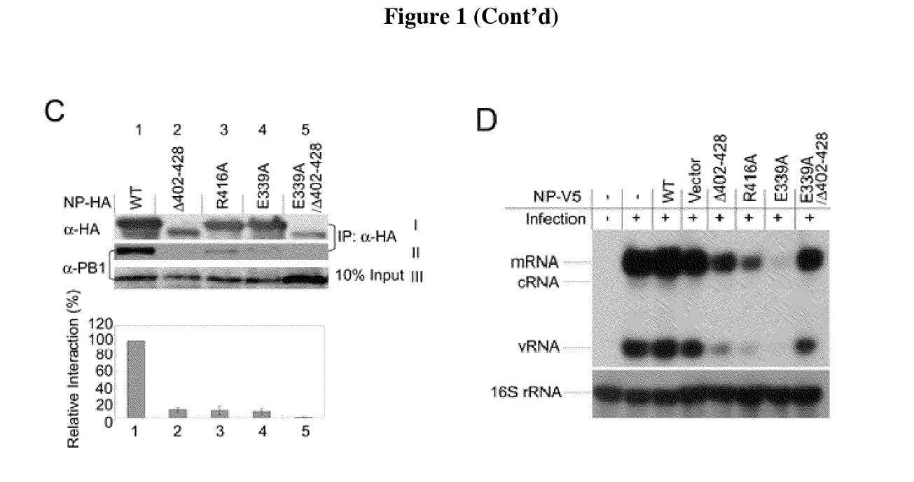

In nature, NP exists predominantly as trimmers, which is essential to influenza virus replication. NP mutants Δ402-428, E339A/R416A, and E339A/Δ402-428 were constructed to study the role of the E339 . . . R416 salt bridge in NP-NP interaction to form trimers. 2×10⁶ HEK293T cells were co-transfected with 8 μg of a plasmid for expressing Flag-tagged WT NP or an HA-tagged NP mutant. Immunoprecipitation (IP) analysis was performed 30 h post transfection using total cell extract by anti-Flag and anti-HA agarose, and visualized by anti-Flag and anti-HA antibodies. As shown in FIG. 1A (rows I and II), the WT NP pulled down R416A (lane 3) and E339A (lane 4) similarly to the control WT NP (lane 1), but it pulled down less of the deletion mutant (Δ402-428, lane 2) and nearly none for the double mutant (E339A/Δ402-428, lane 5). Consistently, rows III and IV of FIG. 1A show that the HA-tagged mutants R416A and E339A can pull down the WT NP with good efficiently (lanes 3 and 4), but the double mutant was unable to pull down WT NP (lane 5).

The binding of E339A and R416A with WT NP was further examined by AUC analyses. WT NP exists predominantly as trimers whereas E339A and R416A exist as monomers. As shown in FIG. 1B, the 1:1 mixture of WT NP with E339A or R416A exists as a mixture of oligomers, demonstrating R416A and E339A bind WT NP to form hetero oligomers, whereas the corresponding mixture with Δ402-428 or E339A/Δ402-428 remains as separate monomer and trimer.

The interaction of R416A and E339A with polymerase basic protein 1 (PB1) of RDRP was examined. 2×10⁶ HEK293T cells were transfected with 8 μg of each plasmid separately (pClneo-NP-HA or pClneo-mutant-HA) for 24 h, then infected with the WSN virus (MOI=0.2) for 12 h. FIG. 1C shows that the HA-tagged wild type NP pulled down PB1 well, all of the NP mutants pulled down little or none. These results demonstrated that R416A and E339A could not bind PB1 because the slightly perturbed hetero complexes with WT NP are unable to further interact with RDRP from the infecting virus.

The inhibition of the transcription-replication activity of RDRP by E339A and R416A was confirmed by the primer extension assay. MDCK cells were infected with the WSN virus at MOI of 2. RNA was isolated from cells 6 h after infection and analyzed by primer extension assays. FIG. 1D show that stable expression of E339A or R416A in MDCK cell lines reduced the synthesis of mRNA and vRNA significantly (the cRNA levels are too low to be detected). The deletion mutant Δ402-428 displayed a smaller effect, and the double mutant E339A/Δ402-428 showed relatively minor or no inhibition.

Taken together, the above results show only E339A and R416A could form hetero complex with WT NP, but the complex was unable to bind the RNA polymerase, leading to inhibition of viral replication. These results demonstrate the importance of the E339 . . . R416 salt bridge in viral survival and establish the salt bridge as a sensitive anti-influenza target.

### (II) NP Fragments Encompassing R416 can Disrupt NP-NP Interaction and Inhibit Viral Replication

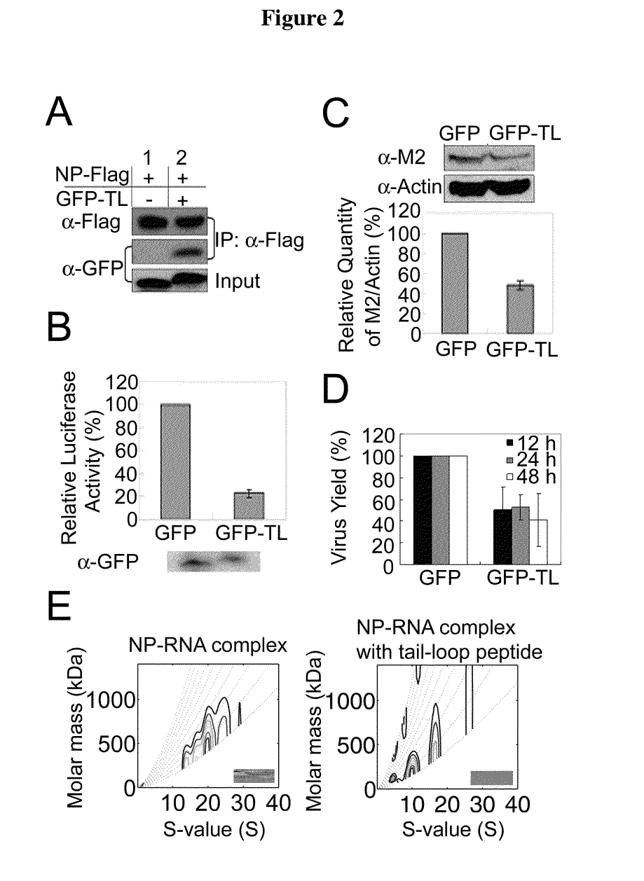

A tail-loop peptide (residues 402-428) of NP was expressed and fused to the Enhanced Green Fluorescent Protein (EGFP) in HEK293T cells and showed that it binds Flag-tagged WT NP by IP assays (FIG. 2A). 2×10⁶ HEK293T cells were co-transfected with 8 μg plasmids of pClneo-NP-Flag in the presence of pEGFP or pEGFP-tail-loop (GFP-TL) plasmid.

The effect of EGFP-tail-loop on the H1N1 polymerase activity was tested by the luciferase-based reporter assays as shown in FIG. 2B. HEK293T cells were co-transfected with pPOLI-Luc-RT, pcDNA-PB1, -PB2, -PA, -NP and with pEGFP (GFP) or GFP-TL plasmid. The expression levels of EGFP and EGFP-tail-loop were determined by western blot analysis using anti-GFP antibodies.

The inhibition effect of EGFP-fused tail-loop peptide (H1N1) on viral replication was demonstrated by transfection of the EGFP-fused tail-loop peptide into HEK293T cells which were subsequently infected with the H1N1 virus. The viral titers of the cell culture supernatant were determined by plaque assays. The relative virus yields were determined 12, 24 and 48 h post infection. As shown in FIG. 2C-D, the EGFP-fused tail-loop peptide was able to slow down the replication of the virus by >50% based on the western blot analyses and the plaque assays.

It was also confirmed by AUC analyses that binding of the tail-loop peptide causes inhibition of the NP oligomerization in the presence of RNA (FIG. 2E). The NP-RNA complex was incubated with synthesized tail loop peptide at a molar ratio of 1:1000.

Example disclosed herein demonstrated that the E339 . . . R416 salt bridge is essential in viral survival and establish the salt bridge as a sensitive anti-influenza target. Peptides encompassing R416 can disrupt NP-NP interaction and inhibit viral replication.

### (III) Inhibition Effects of Peptide Inhibitors Targeting the E339 . . . R416 Salt Bridge.

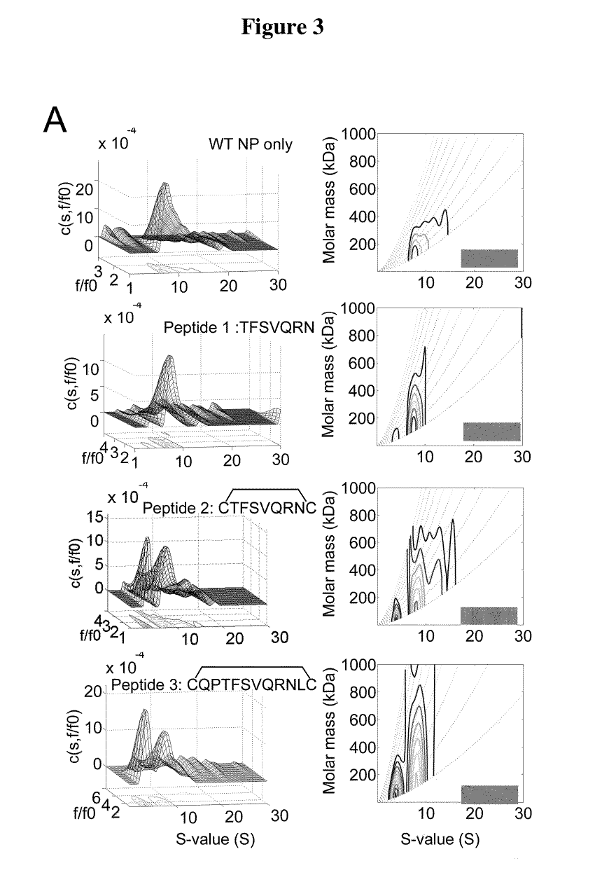

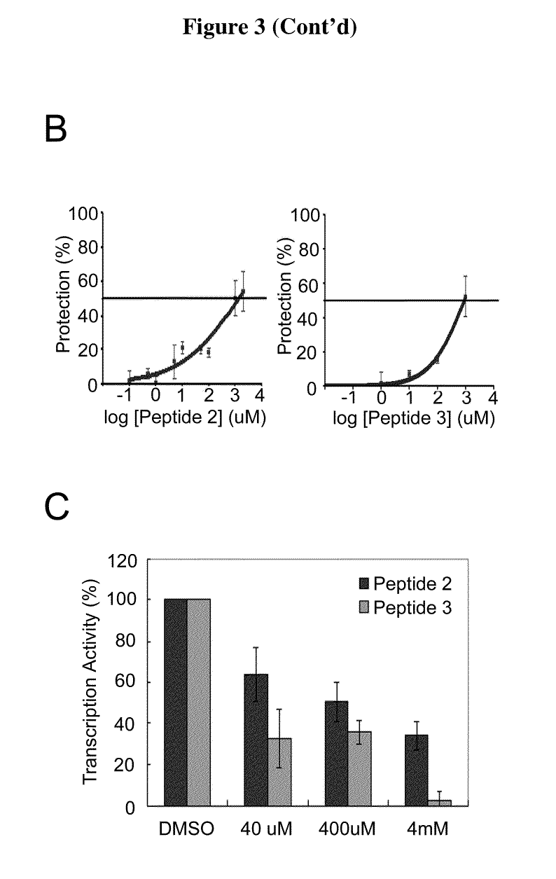

Shorter and cyclic tail-loop peptides of NP were designed and tested for the inhibition effect of the influenza viral replication. As shown in FIG. 3A, a 7-residue peptide (TFDSVQRN, peptide 1; SEQ ID NO:8) from residues 411-417 of the tail-loop disrupted WT NP trimerization only slightly, but the effect was substantially enhanced when the peptide is cyclized to restrict its conformation by adding two Cys residues on both ends (CTFDSVQRNC, peptide 2; SEQ ID NO:2). A slightly larger cyclic peptide (CQPTFSVQRNLC, peptide 3; SEQ ID NO:4) from residues 409-418 of the tail-loop with two Cys residues on both ends, showed a slightly greater effect. AUC analyses demonstrated that disruption of the NP trimer formation is a feasible predictor for the inhibition effect of the influenza viral replication.

The WSN viral yield reduction by cyclic peptides 2 and 3 was analyzed. The MDCK cells were incubated with the peptide (0-2 mM), then inoculated with medium alone or the WSN virus (MOI=0.001) for 48 h at 35° C. The number of metabolically viable cells was determined. The antiviral dose response curves of the peptides (FIG. 3B) showed these peptides were effective in protecting the host cells from viral infection, with the antiviral IC₅₀ values of about 1 mM (Table 2).

TABLE 2
Antiinfluenza IC₅₀ values (μM) of
peptides and small molecule inhibitors

| | | pep- | com- | com- | com- | com- |
|---|---|---|---|---|---|---|
| | peptide 2 | tide 3 | pound 1 | pound 2 | pound 3 | pound 4 |
| IC₅₀ (μM) | 1315 | 904 | 2.7 | 37.5 | 118.4 | 39.7 |
| CC₅₀ (μM)^{†} | >2000 | >2000 | 35.6 | >100 | >100 | >100 |

^{†}CC₅₀ indicated the concentration needed to inhibit 50% growth of MDCK cells in 48 h.

In vitro transcription assay was performed to verify both cyclic peptides 2 and 3 inhibited viral replication by perturbing the RDRP activity. As shown in FIG. 3C, both cyclic peptides 2 and 3 were able to inhibit the influenza viral replication and reduce the viral transcription activity.

### (IV) Inhibition Effects of Small Molecule Inhibitors Targeting the E339 . . . R416 Salt Bridge.

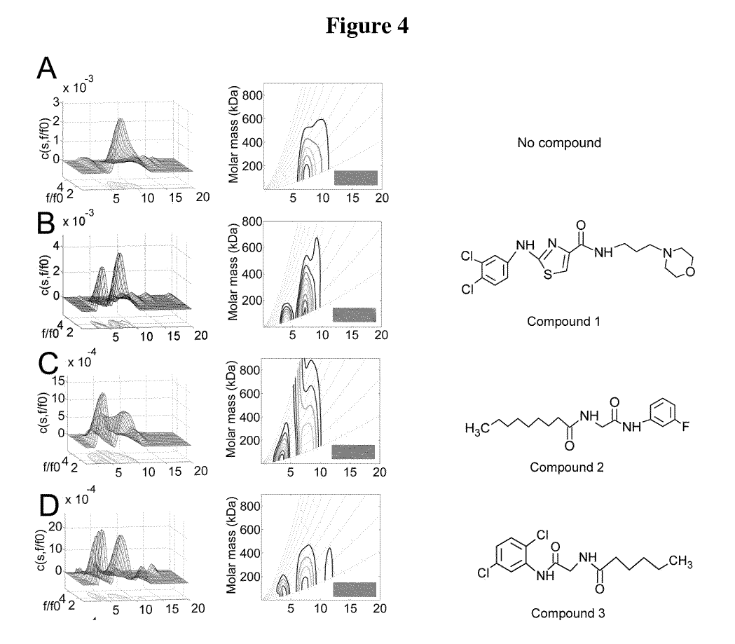

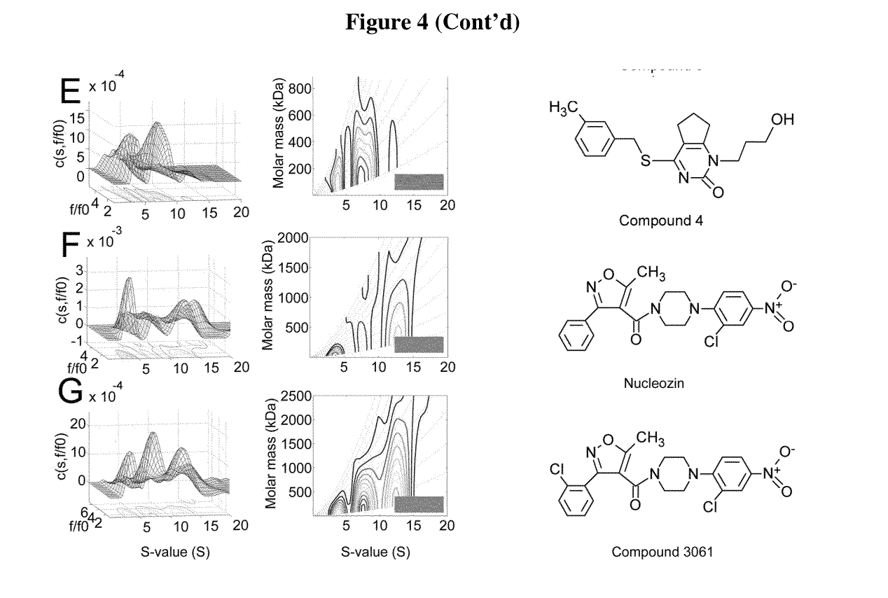

Compounds 1, 2, 3, and 4 were selected from virtual screening a library of 1,775,422 compounds and verified in cell viability antiviral assays. The effects of compounds 1, 2, 3, and 4 for NP WT trimer was analyzed by AUC as shown in FIG. 4A-E. Each sample contained a mixture of 3 μM WT NP with (A) none; (B) 7.5 μM compound 1; (C) 7.5 μM compound 2; (D) 7.5 μM compound 3; (E) 7.5 μM compound 4. The result showed the four compounds are able to disrupt NP trimerization and induce formation of NP monomers. In contrast, nucleozin (compound 788) and compound 3061 caused aggregation of the NP trimer (FIG. 4F-G).

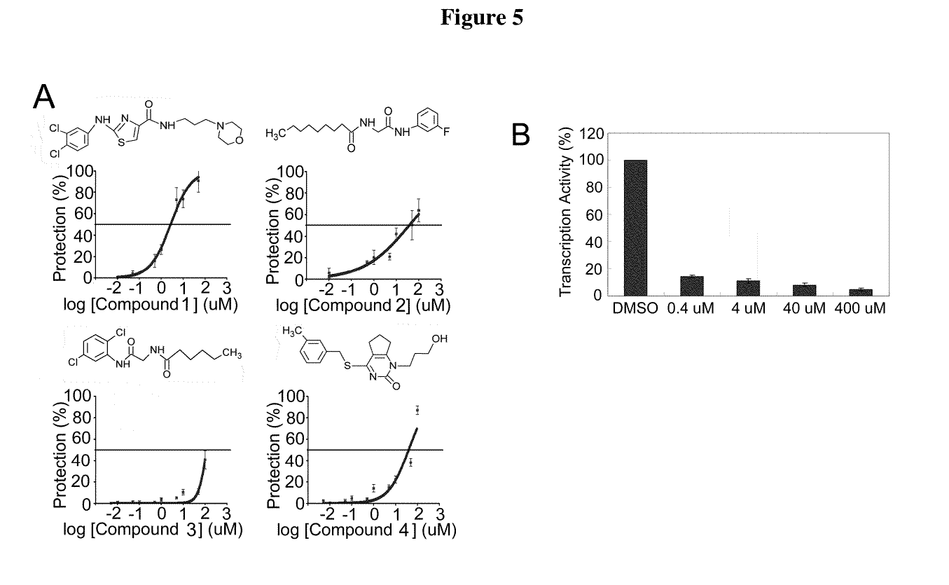

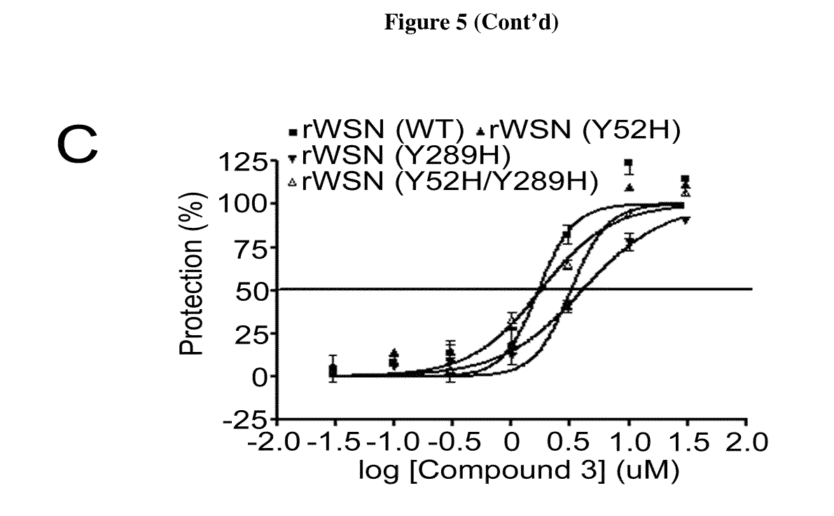

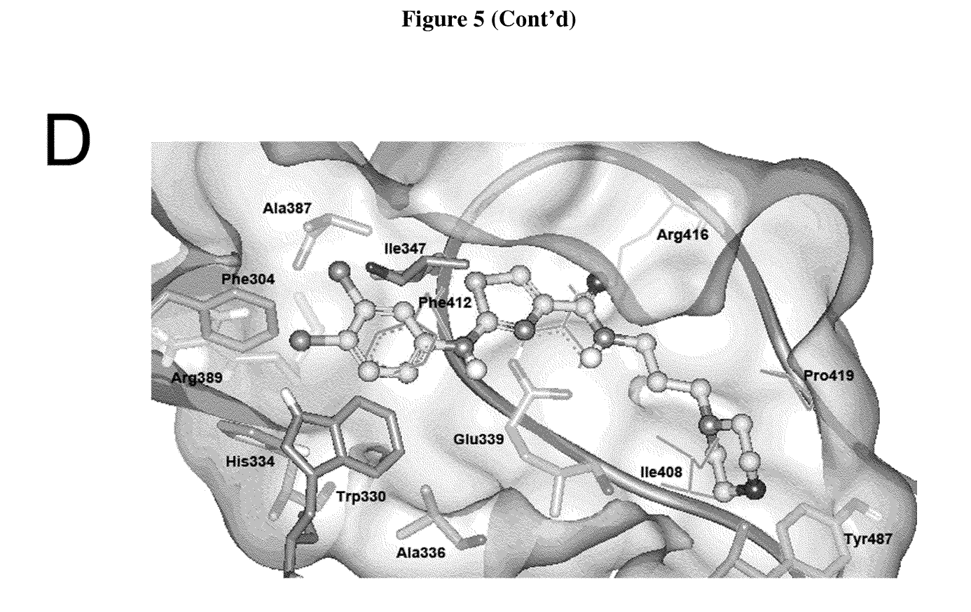

Inhibition effect of compounds 1, 2, 3, and 4 were characterized. FIG. 5A shows the results of antiviral dose response curves. The MDCK cells were incubated with compounds (0-100 μM), then inoculated with medium alone or the WSN virus (MOI=0.001) for 48 h at 35° C. The number of metabolically viable cells was determined.

The in vitro transcription assay of compound 1 was performed as shown in FIG. 5B. The WSN viruses were incubated with compound 1 (0-400 μM) for 1 h at 25° C. The antiviral IC₅₀ values are shown in Table 3.

TABLE 3
Antiinfluenza IC₅₀ values of compound 1 against
recombinant wildtype and mutant strains.

| | Anti-rWSN IC₅₀ (μM)^{†} | | | |
|---|---|---|---|---|
| | parental | NP Y52H | NP Y289H | NP Y52H/Y289H |
| compound 1 | 1.7 | 3.2 | 4.0 | 1.8 |

^{†}Antiinfluenza activities using recombinant virus in WSN background.

### (V) Inhibition Effects of Compound 1 for Drug-Resistant Strains of the Influenza Virus.

Since the E339 . . . R416 salt bridge is highly conserved, it is less likely for the virus to develop resistance against the drugs targeting this specific site. Compound 1 was tested against recombinant wildtype WSN (rWSN), Y52H and Y289H mutant strains, and Y52H/Y289H double mutant. As shown in FIG. 5C, the MDCK cells were incubated with the compound (0-100 μM), then inoculated with medium alone, the rWSN virus, nucleozin-resistant rWSN virus (Y289H mutation at NP), compound 3061-resistant rWSN virus (Y52H mutation at NP), or Y52H/Y289H double mutation rWSN virus (MOI=0.001) at 35° C. After 48 h, the number of metabolically viable cells was determined. The result showed that compound 1 is capable of inhibiting nucleozin- and 3061-resistant strains of the virus.

Table 3 above summarizes the antiinfluenza IC₅₀ values of compound 1 against recombinant wildtype WSN (rWSN), Y52H and Y289H single mutants, and Y52H/Y289H double mutant. strains.

Taken together, the results obtained from this study demonstrated that (a) the salt bridge in a viral NP protein corresponding to the E339 . . . R416 salt bridge in SEQ ID NO:1 is a useful target for treating influenza virus infection and identification of anti-flu agents, and (b) a number of agents thus identified successfully inhibited viral replication, indicating that they are effective agents in treating influenza virus infection.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of identifying an influenza A virus inhibitor, comprising:
contacting a candidate agent with an influenza A virus nucleoprotein, which is in trimer form,
determining disruption of the trimer form of the nucleoprotein, and
assessing whether the candidate agent is an influenza A virus inhibitor, wherein disruption of the trimer form of the nucleoprotein indicates that the candidate agent is an influenza A virus inhibitor.

2. The method of claim 1, wherein the determining step is performed by detecting presence of monomers or oligomers of the nucleoprotein after the contacting step, wherein the oligomers each contain either less than three or more than three NP monomers.

3. The method of claim 2, wherein the presence of the monomers or oligomers of the nucleoprotein is detected by a process comprising:
performing an analytical ultracentrifugation (AUC) assay on the nucleoprotein after the contacting step,
measuring mass distribution of the nucleoprotein, NP; and
comparing the mass distribution with that of the nucleoprotein in trimer form;
wherein a difference between the mass distribution of the nucleoprotein treated with the candidate agent and that of the nucleoprotein in trimer form indicates presence of the monomers or oligomers of the nucleoprotein.

4. A method of inhibiting influenza A virus replication, comprising contacting cells infected with or suspected of being infected an influenza A virus an effective amount of an agent that disrupts a salt bridge in an influenza virus nucleoprotein, wherein the salt bridge corresponds to an E339 . . . R416 salt bridge in SEQ ID NO:1.

5. The method of claim 4, wherein the contacting step is performed by administering the agent to a subject infected with or suspected of being infected with the influenza A virus.

6. The method of claim 5, wherein the agent is administered in an amount effective in treating an infection caused by the influenza A virus.

7. The method of claim 6, wherein the subject has or is suspected of having infection with a wild-type influenza A virus or a mutant influenza A virus that carries a Y289H, Y52H, or Y52H/Y289H mutation in its nucleoprotein.

8. The method of claim 4, wherein the subject has or is suspected of having infection with H1N1, H5N1, or H3N2.

9. The method of claim 4, wherein an agent is a peptide or a compound.

10. The method of claim 9, wherein the agent is a compound selected from the group consisting of:
(a) a compound of Formula (I): 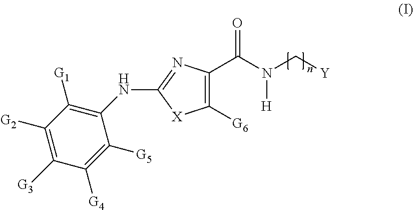
wherein:
each of G₁, G₂, G₃, G₄, G₅ and G₆ is, independently, selected from the group consisting of H, F, Cl, Br, I, OH, O-alkyl (C₁-C₃), NH₂, NH-alkyl (C₁-C₂), NR₂ (R═CH₃ or C₂H₅), NHCOR (R═CH₃ or C₂H₅), N₃, NO₂, alkyl (C₁-C₃), CF₃, phenyl, C≡N, CHO, RCO(R═CH₃ or C₂H₅), CO₂H, CO₂R(R═CH₃ or C₂H₅), CONHR(R═CH₃ or C₂H₅), and SO₃H;
X is selected from the group consisting of S, O, NH and NR(R═CH₃ or C₂H₅);
n is an integer between 1 and 6 inclusive; and
Y is selected from the group comprising phenyl, morpholine, and piperazine;
(b) a compound of Formula (II): 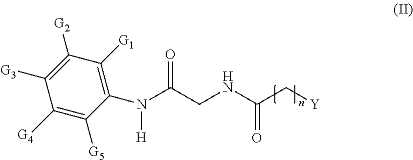
wherein:
each of G₁, G₂, G₃, G₄ and G₅ is, independently, selected from the group consisting of H, F, Cl, Br, I, OH, O-alkyl (C₁-C₃), NH₂, NH-alkyl (C₁-C₂), NR₂ (R═CH₃ or C₂Hs), NHCOR(R═CH₃ or C₂H₅), N₃, NO₂, alkyl (C₁-C₃), CF₃, phenyl, C≡N, CHO, RCO(R═CH₃ or C₂H₅), CO₂H, CO₂R(R═CH₃ or C₂H₅), CONHR(R═CH₃ or C₂Hs), and SO₃H;
n is an integer between 1 and 10 inclusive; and
Y is alkyl or aryl; and
(c) 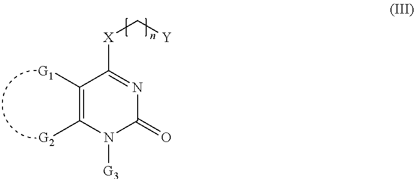
wherein:
each of G₁ and G₂ is, independently, selected from the group consisting of H, F, Cl, Br, I, OH, O-alkyl (C₁-C₃), NH₂, NH-alkyl (C₁-C₂), NR₂ (R═CH₃ or C₂H₅), NHCOR(R═CH₃ or C₂H₅), N₃, NO₂, alkyl (C₁-C₃), CF₃, phenyl, C≡N, CHO, CH₃CO, CO₂H, CO₂R(R═CH₃ or C₂H₅), CONHR(R═CH₃ or C₂H₅), and SO₃H; and G₁ and G₂ optionally is connected to form a 5-7 membered ring;
G₃ is selected from the group consisting of H, alkyl (C₁-C₆), [phenyl]methyl, ω-hydroxyalkyl (C₁-C₄), phenyl, RCO(R═CH₃ or C₂H₅), CO₂R(R═CH₃ or C₂H₅), and CONR₂ (R═CH₃ or C₂H₅);
X is selected from the group consisting of CH₂, S, O, NH and NR(R═CH₃ or C₂H₅);
n is an integer between 1 and 6 inclusive; and
Y is phenyl, morpholine, or piperazine.

11. The method of claim 10, wherein the agent is a compound of Formula (I).

12. The method of claim 11, wherein the compound of Formula (I) is:

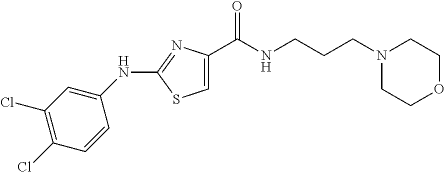 [SMILES: O=C(NCCCN1CCOCC1)c1csc(Nc2ccc(Cl)c(Cl)c2)n1]

13. The method of claim 11, wherein the agent is a compound of Formula (II).

14. The method of claim 13, wherein the compound of Formula (II) is

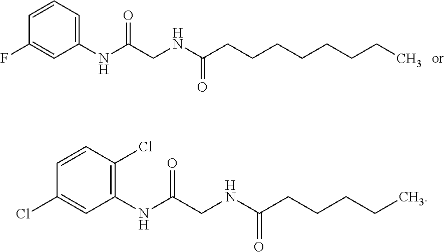 [SMILES: CCCCCCCCC(=O)NCC(=O)Nc1cccc(F)c1 ; CCCCCC(=O)NCC(=O)Nc1cc(Cl)ccc1Cl]

15. The method of claim 10, wherein the agent is a compound of Formula (III).

16. The method of claim 15, wherein the compound of Formula (III) is

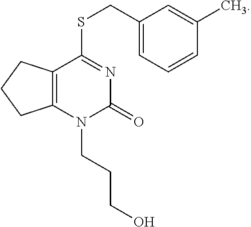 [SMILES: Cc1cccc(CSc2nc(=O)n(CCCO)c3c2CCC3)c1]

17. The method of claim 10, wherein the agent is a peptide that comprises an amino acid sequence at least 80% identical to a fragment of an influenza virus nucleoprotein, the fragment encompassing an Arg residue corresponding to R416 in SEQ ID NO:1.

18. The method of claim 17, wherein the fragment of the influenza virus nucleoprotein comprises a segment corresponding to a region spanning from T411 to N417 in SEQ ID NO:1.

19. The method of claim 17, wherein the peptide comprises two cysteine residues, one flanking one end of the nucleoprotein fragment and the other flanking the other end of the nucleoprotein fragment.

20. The method of claim 19, wherein the peptide comprises the amino acid sequence of CTFSVQRNC (SEQ ID NO:2), CPTFSVQRNLC (SEQ ID NO:3), or CQPTFSVQRNLC (SEQ ID NO:4).

21. The method of claim 19, wherein the peptide is cyclized through a disulfide bond formed between the two cysteine residues.

22. A pharmaceutical composition comprising an agent that disrupts a salt bridge in an influenza virus nucleoprotein and a pharmaceutically acceptable carrier, wherein the salt bridge corresponds to an E339 . . . R416 salt bridge in SEQ ID NO:1, and wherein the agent is a compound or a peptide,
wherein:
(a) the compound is selected from the group consisting of:
(i) a compound of Formula (I): 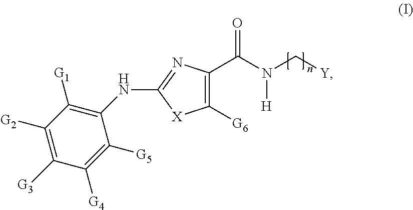
in which
each of G₁, G₂, G₃, G₄, G₅ and G₆ is, independently, selected from the group consisting of H, F, Cl, Br, I, OH, O-alkyl (C₁-C₃), NH₂, NH-alkyl (C₁-C₂), NR₂ (R═CH₃ or C₂H₅), NHCOR (R═CH₃ or C₂H₅), N₃, NO₂, alkyl (C₁-C₃), CF₃, phenyl, C≡N, CHO, RCO(R═CH₃ or C₂H₅), CO₂H, CO₂R(R═CH₃ or C₂H₅), CONHR(R═CH₃ or C₂H₅), and SO₃H;
X is selected from the group consisting of S, O, NH and NR(R═CH₃ or C₂H₅);
n is an integer between 1 and 6 inclusive; and
Y is selected from the group comprising phenyl, morpholine, and piperazine;
(ii) a compound of Formula (II): 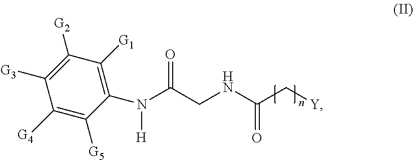
in which
each of G₁, G₂, G₃, G₄ and G₅ is, independently, selected from the group consisting of H, F, Cl, Br, I, OH, O-alkyl (C₁-C₃), NH₂, NH-alkyl (C₁-C₂), NR₂ (R═CH₃ or C₂H₅), NHCOR(R═CH₃ or C₂H₅), N₃, NO₂, alkyl (C₁-C₃), CF₃, phenyl, C≡N, CHO, RCO(R═CH₃ or C₂H₅), CO₂H, CO₂R(R═CH₃ or C₂H₅), CONHR(R═CH₃ or C₂H₅), and SO₃H;
n is an integer between 1 and 10 inclusive; and
Y is alkyl or aryl; and
(iii) a compound of Formula (III) 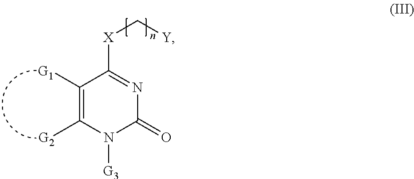
in which
each of G₁ and G₂ is, independently, selected from the group consisting of H, F, Cl, Br, I, OH, O-alkyl (C₁-C₃), NH₂, NH-alkyl (C₁-C₂), NR₂ (R═CH₃ or C₂H₅), NHCOR(R═CH₃ or C₂H₅), N₃, NO₂, alkyl (C₁-C₃), CF₃, phenyl, C≡N, CHO, CH₃CO, CO₂H, CO₂R(R═CH₃ or C₂H₅), CONHR(R═CH₃ or C₂H₅), and SO₃H; and G₁ and G₂ optionally is connected to form a 5-7 membered ring;
G₃ is selected from the group consisting of H, alkyl (C₁-C₆), [phenyl]methyl, ω-hydroxyalkyl (C₁-C₄), phenyl, RCO(R═CH₃ or C₂H₅), CO₂R(R═CH₃ or C₂H₅), and CONR₂ (R═CH₃ or C₂H₅);
X is selected from the group consisting of CH₂, S, O, NH and NR(R═CH₃ or C₂H₅);
n is an integer between 1 and 6 inclusive; and
Y is phenyl, morpholine, or piperazine; and
Wherein:
(b) the peptide comprises an amino acid sequence at least 80% identical to a fragment of an influenza virus nucleoprotein, the fragment encompassing an Arg residue corresponding to R416 in SEQ ID NO:1.


SEQUENCE LISTING (8 sequences)

SEQ ID NO: 1 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTVMAAFSGNTEGRTSDMRTEIIRMMESARPEDVSFQGRGVFELSDEKAASPIVPSFDMSNEGSYFFGDNAEEYDN

SEQ ID NO: 2 - protein, 9 aa, Artificial Sequence
  Synthetic Polypeptide
CTFSVQRNC

SEQ ID NO: 3 - protein, 11 aa, Artificial Sequence
  Synthetic Polypeptide
CPTFSVQRNLC

SEQ ID NO: 4 - protein, 12 aa, Artificial Sequence
  Synthetic Polypeptide
CQPTFSVQRNLC

SEQ ID NO: 5 - DNA, 20 bp, Artificial Sequence
  Synthetic Oligonucleotide
tggactagtgggagcatcat

SEQ ID NO: 6 - DNA, 22 bp, Artificial Sequence
  Synthetic Oligonucleotide
tccagtatggttttgatttccg

SEQ ID NO: 7 - DNA, 20 bp, Artificial Sequence
  Synthetic Oligonucleotide
tactatctctatcgctccaa

SEQ ID NO: 8 - protein, 8 aa, Artificial Sequence
  Synthetic Polypeptide
TFDSVQRN